(19) United States
(12) Patent Application Publication
(10) Pub. No.: US 2020/0357526 A1
(43) Pub. Date: Nov. 12, 2020

(54) SYSTEMS AND METHODS FOR CLINICAL GUIDANCE OF GENETIC TESTING FOR PATIENTS VIA A MOBILE APPLICATION

(71) Applicant: Hygea Precision Medicine, Inc., San Francisco, CA (US)

(72) Inventors: Eyal Odiz, Los Altos Hills, CA (US); Devina Do, San Francisco, CA (US)

(21) Appl. No.: 15/929,475

(22) Filed: May 4, 2020

Related U.S. Application Data
(60) Provisional application No. 62/846,285, filed on May 10, 2019.

(51) Int. Cl. G16H 70/60; G16H 10/60; G16H 50/20; G16H 10/40; G16H 50/70; G16B 20/00

(52) U.S. Cl. CPC G16H 70/60; G16H 10/60; G16B 20/00; G16H 10/40; G16H 50/70; G16H 50/20

(57) ABSTRACT

The present disclosure provides systems and methods for clinical guidance of genetic testing for patients via a mobile application using artificial intelligence. In an aspect, the present disclosure provides a system for enabling an operator to generate a genomic health record of a subject, comprising: a database comprising clinical indications; a communications interface in communication with a computer of the operator; and a computer processor operatively coupled to said database and the communications interface, wherein said computer processor is programmed to (i) receive a request from the computer of the operator to evaluate a clinical indication of the subject, (ii) upon receiving the request, determine genomic data relevant to the clinical indication, and (iii) generate an output, which output comprises a recommendation that said operator have the genomic data obtained for the subject.

### CROSS-REFERENCE

This application claims the benefit of U.S. Provisional Patent Application No. 62/846,285, filed May 10, 2019, which is incorporated by reference herein in its entirety.

### BACKGROUND

Physicians such as primary care physicians and family doctors may not be familiar with genetic testing. In many clinical cases, such physicians may not be aware if a genetic test is needed for a particular subject, what tests to order for the subject, and how to interpret the lab results. As a result, genetic testing may not be used as part of physicians' daily clinical diagnostics procedures for patients.

There are currently more than 74 thousand genetic tests commercially available in the United States, which collectively are clinically relevant to many diseases. For example, genetic tests can be used to determine drugs' effectiveness (pharmacogenomics), pro-active diagnostics (e.g., carrier screening), or to assist with clinical diagnostics of diseases. Since many physicians may not be using these tests in the clinical setting, patents' treatment may not be optimally effective.

### SUMMARY

The present disclosure provides systems and methods for clinical guidance of genetic testing for patients via a mobile application using artificial intelligence. Using systems and methods of the present disclosure, an operator can generate a genomic health record of a subject by evaluating a clinical indication of a subject, determining genomic data relevant to the clinical indication, and recommending genomic data be obtained for the subject. The genomic data may be obtained by performing one or more genetic tests on the subject.

In an aspect, the present disclosure provides a system for enabling an operator to generate a genomic health record of a subject, comprising: a database comprising clinical indications; a communications interface in communication with a computer of said operator; and a mobile computing device comprising a computer processor operatively coupled to said database and said communications interface, wherein said computer processor is programmed to (i) receive a request from said mobile computing device of said operator to evaluate a clinical indication of said subject, (ii) upon receiving said request, determine genomic data relevant to said clinical indication, and (iii) generate an output, which output comprises a recommendation that said operator have said genomic data obtained for said subject. In some embodiments, the system is compatible with the Health Insurance Portability and Accountability Act (HIPAA) and the General Data Protection Regulation (GDPR). In some embodiments, the system is certified by the HITRUST Alliance.

In some embodiments, said operator is a physician. In some embodiments, said computer processor is further programmed to navigate said operator to a clinical view subsequent to receiving said request from said computer of said operator to evaluate said clinical indication. In some embodiments, said computer processor is further programmed to navigate said operator from said clinical view to a disease category view. In some embodiments, said computer processor is further programmed to navigate said operator from said disease category view to a disease view. In some embodiments, said computer processor is further programmed to navigate said operator from said disease view to a genes view. In some embodiments, said genomic data relevant to said clinical indication is determined based at least in part on information of said subject selected from the group consisting of clinical symptoms, patient history, and family history. In some embodiments, said computer processor is further programmed to apply an artificial intelligence algorithm to said clinical indication and said information of said subject to determine said genomic data relevant to said clinical indication. In some embodiments, said recommendation comprises one or more genetic tests to obtain said genomic data for said subject. In some embodiments, said artificial intelligence algorithm comprises an optimization based on a constraint comprising one or more of: a number of said one or more genetic tests, a cost of said one or more genetic tests, an accuracy of said evaluation of said clinical indication, and a confidence of said evaluation of said clinical indication. In some embodiments, said recommendation is customized to a hospital population. In some embodiments, said hospital population is based on a geographical location. In some embodiments, said mobile computing device is selected from the group consisting of a smart phone, a tablet computer, a smart watch, and a wearable computer. In some embodiments, said mobile computing device comprises a smart phone. In some embodiments, said mobile computing device comprises a tablet computer. In some embodiments, said mobile computing device comprises a smart watch. In some embodiments, said mobile computing device comprises a wearable computer.

In another aspect, the present disclosure provides a method for enabling an operator to generate a genomic health record of a subject, comprising: (a) receiving a request from said operator via a mobile application to evaluate a clinical indication of said subject; (b) upon receiving said request, determining genomic data relevant to said clinical indication; and (c) generating an output, which output comprises a recommendation that said operator have said genomic data obtained for said subject. In some embodiments, the method is compatible with the Health Insurance Portability and Accountability Act (HIPAA) and the General Data Protection Regulation (GDPR). In some embodiments, the method is certified by the HITRUST Alliance.

In some embodiments, said operator is a physician. In some embodiments, the method further comprises navigating said operator to a clinical view subsequent to receiving said request from said computer of said operator to evaluate said clinical indication. In some embodiments, the method further comprises navigating said operator from said clinical view to a disease category view. In some embodiments, the method further comprises navigating said operator from said disease category view to a disease view. In some embodiments, the method further comprises navigating said operator from said disease view to a genes view. In some embodiments, the method further comprises determining said genomic data relevant to said clinical indication based at least in part on information of said subject selected from the group consisting of clinical symptoms, patient history, and family history. In some embodiments, the method further comprises applying an artificial intelligence algorithm to said clinical indication and said information of said subject to determine said genomic data relevant to said clinical indication. In some embodiments, said recommendation comprises one or more genetic tests to obtain said genomic data for said subject. In some embodiments, said artificial intelligence algorithm comprises an optimization based on a constraint comprising one or more of: a number of said one or more genetic tests, a cost of said one or more genetic tests, an accuracy of said evaluation of said clinical indication, and a confidence of said evaluation of said clinical indication. In some embodiments, said recommendation is customized to a hospital population. In some embodiments, said hospital population is based on a geographical location. In some embodiments, said mobile application is executed by a computer processor of a mobile computing device. In some embodiments, said mobile computing device is selected from the group consisting of a smart phone, a tablet computer, a smart watch, and a wearable computer. In some embodiments, said mobile computing device comprises a smart phone. In some embodiments, said mobile computing device comprises a tablet computer. In some embodiments, said mobile computing device comprises a smart watch. In some embodiments, said mobile computing device comprises a wearable computer.

In another aspect, the present disclosure provides a method for enabling a physician to generate genetic test results for a patient, comprising: (a) receiving a clinical indication from said physician via a mobile application; (b) determining genomic data relevant to said clinical indication; and (c) making recommendations of genetic tests relevant to said clinical indication from said physician based at least in part on said genomic data. In some embodiments, the method is compatible with the Health Insurance Portability and Accountability Act (HIPAA) and the General Data Protection Regulation (GDPR). In some embodiments, the method is certified by the HITRUST Alliance. In some embodiments, said mobile application is executed by a computer processor of a mobile computing device. In some embodiments, said mobile computing device is selected from the group consisting of a smart phone, a tablet computer, a smart watch, and a wearable computer. In some embodiments, said mobile computing device comprises a smart phone. In some embodiments, said mobile computing device comprises a tablet computer. In some embodiments, said mobile computing device comprises a smart watch. In some embodiments, said mobile computing device comprises a wearable computer.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The patent or application file contains at least one drawing executed in color. Copies of this patent or patent application publication with color drawings will be provided by the Office upon request and payment of the necessary fee. The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 illustrates an example of an architecture of a graphical genome system, in accordance with disclosed embodiments.

FIG. 2 illustrates an example of a typical geneticist workflow, in accordance with disclosed embodiments.

FIG. 3 illustrates an example of a navigator diagnostic workflow, in accordance with disclosed embodiments.

FIG. 4 illustrates an example of a navigator pharmacogenetics workflow, in accordance with disclosed embodiments.

FIG. 5 illustrates an example of a hereditary breast cancer report, which includes results of genetic analysis of a breast cancer gene panel with color coding, and reviewable events, in accordance with disclosed embodiments.

FIG. 6 illustrates an example of a hereditary breast cancer report, which includes results of genetic analysis of a breast cancer gene panel with color coding, in accordance with disclosed embodiments.

FIG. 7 illustrates an example of how gene size can be visualized to reflect probability and quality of data in the genome bank.

FIG. 8 illustrates a computer system that is programmed or otherwise configured to implement methods provided herein.

FIG. 9 illustrates an example of an architecture of a graphical genome system, in accordance with disclosed embodiments.

FIG. 10A-10C illustrates an example of a graphical genome system, in accordance with disclosed embodiments, wherein machine learning-based gene panel selections are customized to a hospital population.

FIGS. 11A-11D illustrate examples of a navigator software of a graphical genome system for use by clinical providers, including a view of a list of clinical pathways (FIG. 11A), views of a proprietary genes panel (FIGS. 11B-11C), and a view of a list of curated lab panels (FIG. 11D), in accordance with disclosed embodiments.

FIGS. 12A-12D illustrate examples of a navigator software of a graphical genome system for use by ordering clinical providers, including a view of a test order history (FIG. 12A), a view of clinical guidances for different diseases or disorders (FIG. 12B), and views of a list of positive genes (FIGS. 12C-12D), in accordance with disclosed embodiments.

FIG. 13 illustrates an example of the design and functionality of a navigator software of a graphical genome system for use with pharmacogenomic data, in accordance with disclosed embodiments.

FIGS. 14A-14E illustrate an example of screenshots of a mobile application to provide clinical guidance of genetic testing for patients, including a view of the main menu for the patient (FIG. 14A), a view of the medication wallet for the patient (FIG. 14B), a view of a specific medication for the patient (FIG. 14C), a view of a dosage of a medication for the patient (FIG. 14D), and a view of test orders for the patient (FIG. 14E), in accordance with disclosed embodiments.

FIGS. 15A-15D illustrate an example of screenshots of a mobile application to provide clinical guidance of genetic testing for patients, including a view of the family members network for the patient (FIG. 15A), a view of the support groups for the patient (FIG. 15B), a view of a specific support group for the patient (FIG. 15C), and a view of a specific discussion in a support group for the patient (FIG. 15D), in accordance with disclosed embodiments.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Physicians such as primary care physicians and family doctors may not be familiar with genetic testing. In many clinical cases, such physicians may not be aware if a genetic test is needed for a particular subject, what tests to order for the subject, and how to interpret the lab results. As a result, genetic testing may not be used as part of physicians' daily clinical diagnostics procedures for patients.

There are currently more than 74 thousand genetic tests commercially available in the United States, which collectively are clinically relevant to many diseases. For example, genetic tests can be used to determine drugs' effectiveness (pharmacogenomics), pro-active diagnostics (e.g., carrier screening), or to assist with clinical diagnostics of diseases. Since many physicians may not be using these tests in the clinical setting, patents' treatment may not be optimally effective.

The present disclosure provides systems and methods for clinical guidance of genetic testing for patients via a mobile application using artificial intelligence. Using systems and methods of the present disclosure, an operator can generate a genomic health record of a subject by evaluating a clinical indication of a subject, determining genomic data relevant to the clinical indication, and recommending genomic data be obtained for the subject. The genomic data may be obtained by performing one or more genetic tests on the subject. The present disclosure further provides systems and methods for hospitals to track and analyze genomic health records across a population of patients (e.g., for population profiling and tracking of metrics such as total genetic test spend for a population of patients).

The present disclosure provides a system to provide guidance to physicians regarding how to incorporate genetic tests into their clinical workflows. Given a patient's symptoms and a clinical guidance, the system may be configured to suggest to the physician if genetic tests are needed. If so, then the system may then determine a set of necessary tests. The system may allow physicians to order the needed genetic tests. In addition, the system may then represent the test results in a visual manner that is easy to understand and interpret, thereby facilitating the physician's clinical decisions regarding subsequent necessary clinical actions.

The system may perform one or more of the following functions: (i) given a patient's symptoms and a clinical guidance, recommend (e.g., using artificial intelligence optimization) one or more genetic tests that may be clinically necessary or helpful toward a clinical diagnostic or prognostic determination; (ii) allow physicians to order the needed genetic tests: (iii) visually display the test results to facilitate the physician's understanding and interpretation and to take necessary subsequent clinical actions; and (iv) store and manage patients' raw data and lab reports.

In some cases, clinical providers may face challenges such as limited genetic knowledge, which may result in ordering a wrong test or having to refer to genetics experts. Using systems and methods of the present disclosure, clinical providers can use clinical support software that helps them order the most effective test. In other cases, clinical providers may face challenges such as difficulty in comparing between laboratories offering tests, which can require logging into multiple labs to compare cost, turnaround time, reimbursement, quality, etc. Using systems and methods of the present disclosure, clinical providers can use one log-in that gives providers access to all labs for comparison. In other cases, clinical providers may face challenges such as difficulty in understanding what action to take, in part because genetic test results are long and complicated. Using systems and methods of the present disclosure, clinical providers can use clinical support software that visualizes test results to highlight actionable data.

In some cases, patients may face challenges such as doctors prescribing them medication that does not seem to work for him or her. Using systems and methods of the present disclosure, patients can see which medications will work for him or her and which ones he or she should avoid. In other cases, patients may face challenges such as lack of an easy way to share their test results and medication data with providers and family members. Using systems and methods of the present disclosure, patients can store and test results and medication data with anyone they choose using the Navigator portal. In other cases, patients may face challenges such as wanting to be a part of a group with people who have similar illnesses and not having a good source for information on new clinical trials. Using systems and methods of the present disclosure, patients can receive the opportunity to join support groups and receive information on new clinical trials.

In an aspect, the present disclosure provides a system for enabling an operator to generate a genomic health record of a subject, comprising: a database comprising clinical indications; a communications interface in communication with a computer of said operator; and a computer processor operatively coupled to said database and said communications interface, wherein said computer processor is programmed to (i) receive a request from said computer of said operator to evaluate a clinical indication of said subject, (ii) upon receiving said request, determine genomic data relevant to said clinical indication, and (iii) generate an output, which output comprises a recommendation that said operator have said genomic data obtained for said subject. In some embodiments, the system is compatible with the Health Insurance Portability and Accountability Act (HIPAA) and the General Data Protection Regulation (GDPR). In some embodiments, the system is certified by the HITRUST Alliance.

In some embodiments, said operator is a physician. In some embodiments, said computer processor is further programmed to navigate said operator to a clinical view subsequent to receiving said request from said computer of said operator to evaluate said clinical indication. In some embodiments, said computer processor is further programmed to navigate said operator from said clinical view to a disease category view. In some embodiments, said computer processor is further programmed to navigate said operator from said disease category view to a disease view. In some embodiments, said computer processor is further programmed to navigate said operator from said disease view to a genes view. In some embodiments, said genomic data relevant to said clinical indication is determined based at least in part on information of said subject selected from the group consisting of clinical symptoms, patient history, and family history. In some embodiments, said computer processor is further programmed to apply an artificial intelligence algorithm to said clinical indication and said information of said subject to determine said genomic data relevant to said clinical indication. In some embodiments, said recommendation comprises one or more genetic tests to obtain said genomic data for said subject. In some embodiments, said artificial intelligence algorithm comprises an optimization based on a constraint comprising one or more of: a number of said one or more genetic tests, a cost of said one or more genetic tests, an accuracy of said evaluation of said clinical indication, and a confidence of said evaluation of said clinical indication.

In another aspect, the present disclosure provides a method for enabling an operator to generate a genomic health record of a subject, comprising: (a) receiving a request from said operator to evaluate a clinical indication of said subject; (b) upon receiving said request, determining genomic data relevant to said clinical indication; and (c) generating an output, which output comprises a recommendation that said operator have said genomic data obtained for said subject. In some embodiments, the method is compatible with the Health Insurance Portability and Accountability Act (HIPAA) and the General Data Protection Regulation (GDPR). In some embodiments, the method is certified by the HITRUST Alliance.

In some embodiments, said operator is a physician. In some embodiments, the method further comprises navigating said operator to a clinical view subsequent to receiving said request from said computer of said operator to evaluate said clinical indication. In some embodiments, the method further comprises navigating said operator from said clinical view to a disease category view. In some embodiments, the method further comprises navigating said operator from said disease category view to a disease view. In some embodiments, the method further comprises navigating said operator from said disease view to a genes view. In some embodiments, the method further comprises determining said genomic data relevant to said clinical indication based at least in part on information of said subject selected from the group consisting of clinical symptoms, patient history, and family history. In some embodiments, the method further comprises applying an artificial intelligence algorithm to said clinical indication and said information of said subject to determine said genomic data relevant to said clinical indication. In some embodiments, said recommendation comprises one or more genetic tests to obtain said genomic data for said subject. In some embodiments, said artificial intelligence algorithm comprises an optimization based on a constraint comprising one or more of: a number of said one or more genetic tests, a cost of said one or more genetic tests, an accuracy of said evaluation of said clinical indication, and a confidence of said evaluation of said clinical indication.

In another aspect, the present disclosure provides a system to provide guidance to physicians regarding how to incorporate genetic tests into their clinical workflows. Given a set of a patient's symptoms and a clinical guidance (e.g., provided by a physician or other clinician), the system may be configured to generate a recommendation to a physician in a hospital regarding whether genetic tests are needed for the patient. If so, the system may then generate a recommendation of a set of necessary tests to be prescribed to the patient. The system may allow physicians in a hospital to order the recommended genetic tests, given the patient's symptoms and clinical guidance. In addition, the system may then represent the test results in a visual manner that is easy to understand and interpret, thereby facilitating the physician's clinical decisions regarding subsequent necessary clinical actions for the patient. In some embodiments, the system allows hospitals to track and analyze across a population of patients (e.g., for population profiling and tracking of metrics such as total genetic test spend for a population of patients). In some embodiments, the system is compatible with the Health Insurance Portability and Accountability Act (HIPAA) and the General Data Protection Regulation (GDPR). In some embodiments, the system is certified by the HITRUST Alliance.

In another aspect, the present disclosure provides a method for enabling a physician to generate genetic test results for a patient, comprising: (a) receiving a clinical indication from said physician; (b) determining genomic data relevant to said clinical indication; and (c) making recommendations of genetic tests relevant to said clinical indication from said physician based at least in part on said genomic data. In some embodiments, the method is compatible with the Health Insurance Portability and Accountability Act (HIPAA) and the General Data Protection Regulation (GDPR). In some embodiments, the method is certified by the HITRUST Alliance.

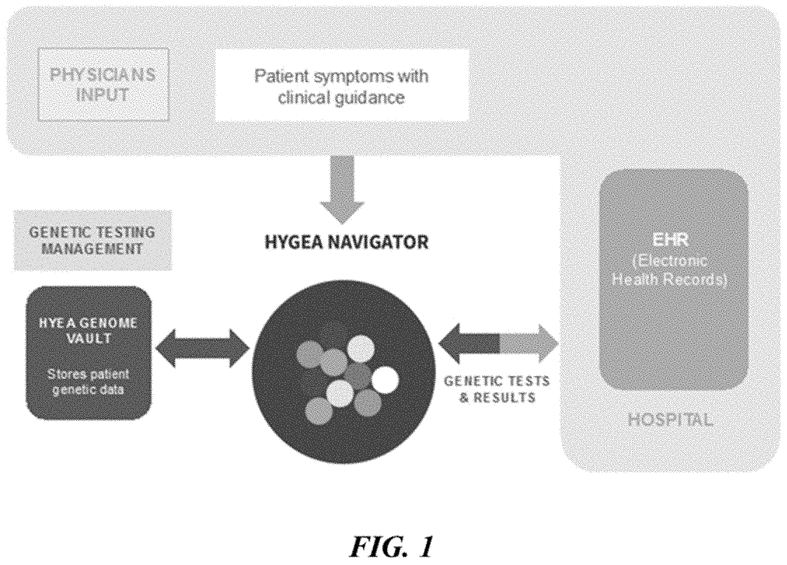

The system for enabling an operator to generate a genomic health record of a subject (e.g., graphical genome system) may comprise two components: a graphical navigator and a genome vault (database). FIG. 1 illustrates an example of an architecture of a graphical genome system, in accordance with disclosed embodiments. A graphical navigator that is clinically aware provides guidance to physicians regarding how to manage genetic tests in a clinical workflow. The navigator connects to a genome database that stores patients' sequencing data and lab reports, and to the electronic health record (EHR) of the hospital where the patients are receiving clinical care. Physicians can run pro-active and symptomatic scenarios through the navigator.

The graphical navigator may comprise an algorithm that combines artificially intelligence-based optimization with powerful graphical representations of clinical knowledge and the relevant genomic information. These graphical representations may provide guidance to physicians through the workflow needed to order and manage genetic tests. The navigator may connect to the Vault and to the EHR (Electronic Health Record) and that is how physicians can access it.

The genome vault may comprise a cloud database that stores patients' raw sequencing data and lab reports. Patients may be able to open an account on their name and deposit into the vault their sequencing raw data and lab reports. In some embodiments, patients can grant physicians access to the data so physicians can leverage it to provide better care and share with other physicians. In some embodiments, patients may share their data with family members. In a further embodiment, patients may grant access to their data to family members thereby allowing family members to access and use the data to provide better health care to the patient. In some embodiments, patients may grant access to their data to a nominated individual thereby allowing the nominated individual to access and use the data to provide better health care to the patient. In a further embodiment, the nominated individual is a spouse, a family member, a friend, a caretaker, an assistant, an aide, or an individual granted power of attorney by the patient. In some embodiments, patients may revoke a previously granted access to their data from the family member or nominated individual. In some embodiments, the family member or nominated individual may grant or revoke access to the patient's data to a physician, clinician, nurse, genetic counselor, or other health care provider.

The navigator may be configured to receive a patient's specific data such as: clinical symptoms, physician clinical guidance, patient history (HX) and family history (HX). The navigator may apply artificial intelligence-based algorithms to the patient's data to compare the data with its knowledge base to identify disease categories and inheritance patterns. The navigator may be configured to, based on the identified disease categories and inheritance patterns, propose one or more gene panels as necessary genetic tests. The more accurately the system can match the patient data with patterns in its knowledge base, the more effective the set of identified genetic tests is expected to be (e.g., a smaller gene panel with higher accuracy).

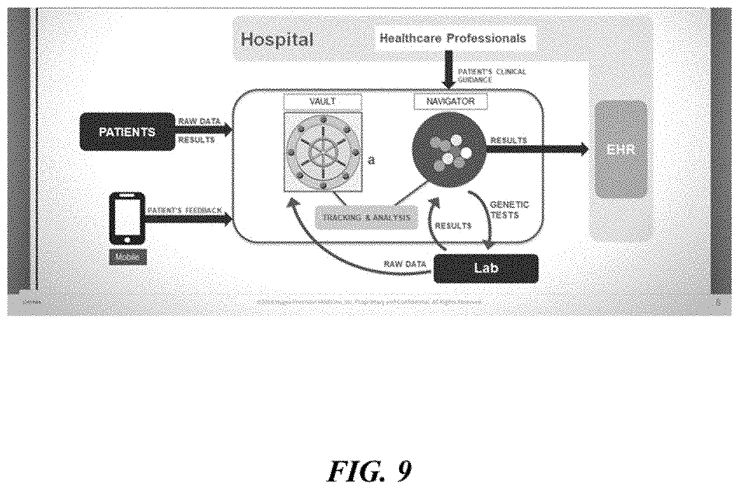

FIG. 9 illustrates another example of an architecture of a graphical genome system, in accordance with disclosed embodiments. A graphical navigator that is clinically aware provides guidance to physicians regarding how to manage genetic tests in a clinical workflow. The navigator connects to a genome database that stores each patient's sequencing data and lab reports, and to the electronic health record (EHR) of the hospital where each patient is receiving clinical care. Physicians can run proactive and symptomatic scenarios using the navigator. The graphical genome system is also connected to a patient's mobile device (e.g., cell phone, tablet computer, laptop computer, desktop computer, smart watch). In one embodiment, patients can use their mobile device to navigate and operate the graphical genome system (e.g., managing access to their data, providing feedback such as symptoms, and viewing reports and summaries of their data). The patients may navigate and operate the graphical genome system using a graphical user interface (GUI).

The graphical navigator may comprise an algorithm that combines artificial intelligence-based optimization with powerful graphical representations of clinical knowledge and the relevant genomic information. These graphical representations may provide guidance to physicians through the workflow needed to order and manage genetic tests. The navigator may connect to the Vault and to the EHR (Electronic Health Record) thereby allowing physicians to access it.

The genome vault may comprise a cloud database that stores patients' raw sequencing data and lab reports. The genome vault may also store patients' feedback provided from their mobile device. Patients may be able to open an account on their name and deposit into the vault (e.g. by uploading) their sequencing raw data and lab reports. In some embodiments, patients can grant physicians access to the data so physicians can leverage it to provide better care and share with other physicians. In some embodiments, patients may share their data with family members or nominated individuals.

The navigator may be configured to receive a patient's specific data such as: clinical symptoms, physician clinical guidance, patient history (HX) and family history (HX). The navigator may apply artificial intelligence-based algorithms to the patient's data to compare the data with its knowledge base to identify disease categories and inheritance patterns. The navigator may be configured to, based on the identified disease categories and inheritance patterns, generate a recommendation of one or more gene panels as necessary genetic tests for the patient. The more accurately the system can match the patient data with patterns in its knowledge base, the more effective the set of identified genetic tests is expected to be (e.g., a smaller gene panel with higher accuracy of obtaining a diagnosis, prognosis, treatment selection, treatment efficacy, treatment monitoring, risk assessment, or other assessment of a patient's health or disease or disorder state).

The term “subject,” as used herein, generally refers to a human or an animal (e.g., a non-human mammal, ape, monkey, chimpanzee, reptile, amphibian, or bird). The subject may be a person with a disease or disorder, or a person that is suspected of having the disease or disorder, or a person that does not have or is not suspected of having the disease or disorder. The disease or disorder may be an infectious disease, an immune disorder or disease, a cancer, a genetic disease, a degenerative disease, a lifestyle disease, an injury, a rare disease, or an age-related disease. The infectious disease may be caused by bacteria, viruses, fungi, and/or parasites.

The subject may be a person with a disease or disorder, or a person that is suspected of having the disease or disorder, or a person that does not have or is not suspected of having the disease or disorder. The disease or disorder may be an infectious disease, an immune disorder or disease, a cancer, a genetic disease, a degenerative disease, a lifestyle disease, an injury, a rare disease or an age related disease. The infectious disease may be caused by bacteria, viruses, fungi and/or parasites.

A physician may generate a clinical indication from one or more initial clinical tests or information. For example, a subject may be present at a clinic with a presence of one or more symptoms, outcomes of general tests, family history, or a combination thereof. A plurality of different genetic tests may be performed for the subject based on the clinical indication. A genetic test may be performed before and/or after treatment of a subject with a disease or disorder. A genetic test may be performed during a treatment or a treatment regime. Examples of a genetic test may include tests for hereditary risk of diseases or disorders such as cardiomyopathy, epilepsy, and progressive ataxia.

In general, physicians may find it challenging to select a small set of genetic tests from among a larger set of commercially available genetic tests to order for a given subject given a clinical indication. For example, if a physician guidance indicates a movement disorder such as Parkinson disease, there may be many (e.g., a few dozen such as about 36, according to GTR) genetic tests available for such a disorder, depending on the type of sequencing and analysis done and the lab that is performing the test. Therefore, reducing a large set of genetic tests to few tests (e.g., no more than 1, no more than 2, no more than 3, no more than 4, or no more than 5) may be a complex problem that requires optimization techniques. By incorporating patient information such as age, history, ethnicity, etc., an artificial intelligence-based optimization may help reduce the number of tests needed to effectively determine a clinical decision. By combining genetic data and clinical data, the physician can provide better diagnostics and better care.

Current methods of providing clinical reports of a genetic test may be used by laboratories to communicate genetic test results back to doctors. In the cases that a genetic test is ordered, the physician may order the genetic test from a laboratory and receive back a report in paper or electronic format (e.g., PDF). The report may be faxed back to the physician after he or she spends significant times on the phone with the laboratory to coordinate the delivery of the report. In many cases, the laboratory report contains detailed information and is hard to interpret and understand by physicians. The physician may scan the paper report into his or her computer, but there may be no process regarding how to share such data with other doctors (e.g., specialists) or clinics. As a result, the genetic testing results may be forgotten or otherwise not incorporated into a clinical decision after a short period of time. A faxed clinical report provided by a laboratory may appear fuzzy and may be difficult to read. The report may present results of a deletion/duplication analysis of a multiple-gene panel that contains elaborate information requiring an experienced user to interpret and understand. The report may be obtained by the patient's doctor after about an hour of communication over the phone, and may contain variants that may have been detectable in parents on carrier screening.

The present disclosure provides a graphical genome system that may advantageously streamline the information management of genetic tests and laboratory reports in a clinical setting. The graphical genome system may include a navigator configured to provide guidance to physicians regarding how to order and manage genetic tests. Lab reports may be stored in a genomic database of the graphical genome system together with the patient's raw data, and the genomic database may be configured to be accessible by any physician who has access to the genomic database (genome bank). The reports may be digitized and can be viewed graphically with actionable parts highlighted.

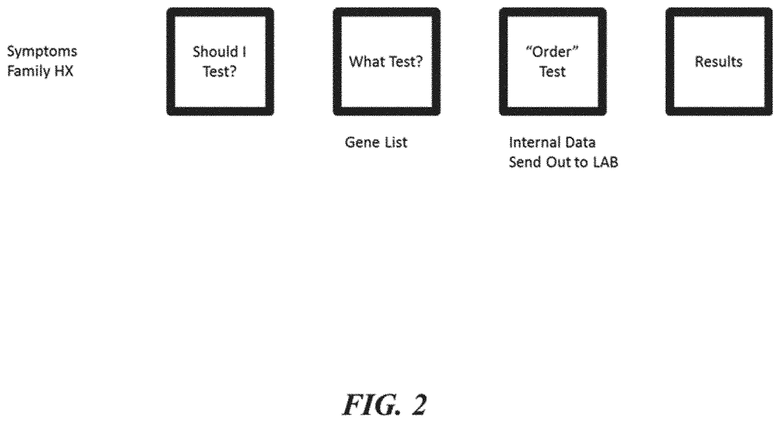

FIG. 2 illustrates an example of a typical geneticist workflow, in accordance with disclosed embodiments. Given a patient's symptoms and family medical history, a geneticist may proceed through the following workflow.

First, the geneticist may determine whether genetic testing is necessary and whether to use genetic testing at all. Such a determination may be made based on the patient information and patient and family history. For example, there may be many known (pre-curated) cases in which genetic tests are necessary to make a clinical decision (e.g., diagnosis, prognosis, or treatment selection).

Next, after the geneticist has determined that genetic testing is necessary, then the geneticist may determine what tests to order for the given subject (patient). A set of one or more genetic tests may be identified based on the genes that are related to the symptoms. For example, for hereditary breast cancer screening, a BRCA1/BRCA2 panel may be ordered. For a given clinical guidance, there may be many genetic tests commercially available, depending on the type of sequencing and the lab. In such cases, artificial intelligence-based optimization can narrow down the initial set of genetic tests to an optimal reduced set of tests (e.g., no more than 1, no more than 2, no more than 3, no more than 4, or no more than 5). Such an optimal reduced set of sets may comprise the recommended genetic tests.

The navigator may emulate the typical geneticist workflow depicted in FIG. 2 in a simple graphical way to help guide physicians through the process.

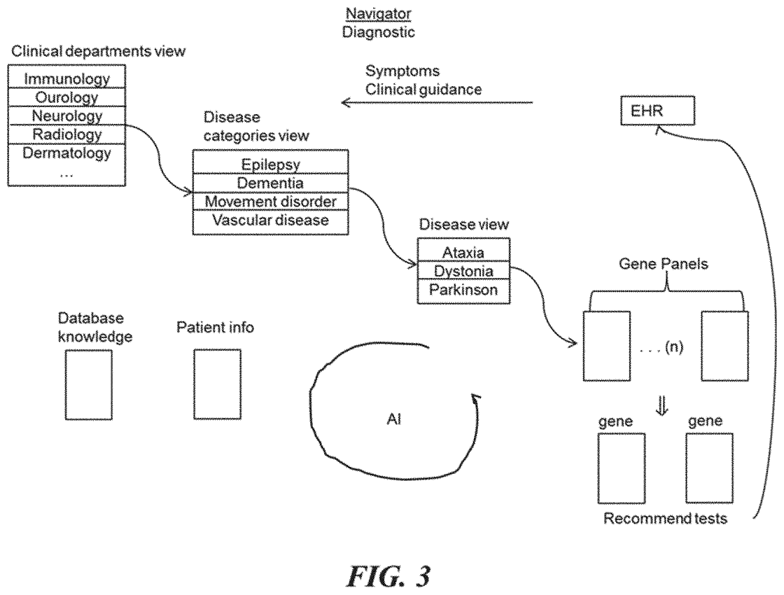

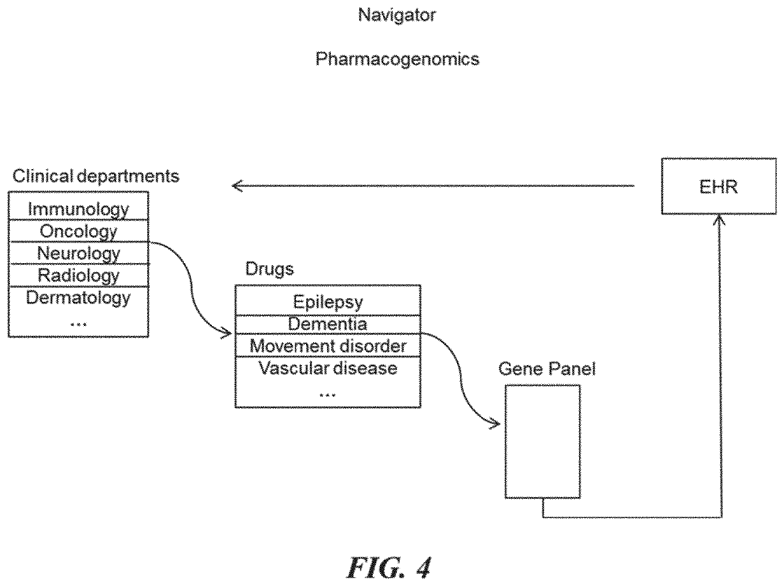

Physicians can use the navigator for clinical diagnosis and/or for drug analysis. For example, FIG. 3 illustrates an example of a navigator diagnostic workflow, in accordance with disclosed embodiments. As another example, FIG. 4 illustrates an example of a navigator pharmacogenetics workflow, in accordance with disclosed embodiments.

The navigator may include a clinical view and a genes view. The clinical view may guide the physician based on the clinical guidance he or she provides. For example, the first clinical view may display a number of different clinical departments, such as: Cardiology, Immunology, Oncology, Neurology, Dermatology, etc. Suppose that the physician chooses Neurology as the clinical department in the clinical view. Then the navigator may generate a disease view that displays a number of different disease categories, such as: Epilepsy, Vascular diseases, White matter diseases, Movement disorders, Dementia, etc. Suppose that the patient's symptoms are related to movement disorder and that the physician suspects Parkinson disease. Then the physician can select Movement disorders as the disease category in the disease view, and the navigator may generate a disease view panel that displays a number of different diseases, such as: Parkinson, Ataxia, Dystonia, etc., from which the physician can select Parkinson.

After the physician selects a disease from the disease view panel, the navigator may generate a view of a gene panel and display a set of one or more genes that, if mutated, can cause hereditary effects of Parkinson. For example, such a set of genes may include: LRPK2, PARK7, PINK1, PRKN, SNCA, GBA, UCHL1, etc.

The navigator may generate a genes view, which can display the recommended genetic tests. After the genetic tests have been ordered by the physician and performed for the subject, the same view may be then populated with the corresponding test results. For the recommended genetic test, the genes to be tested may be displayed. The genes will be annotated with a size that reflects the probability that a mutation in that gene can trigger the disease.

Genetic test results may be displayed with annotations on the same view. The genes may be displayed with visuals of color, size, crosses, and hyperlinks to commentary. The navigator display may be configured in such a way to provide an easy way for the physician to interpret the results, determine the quality of the data, the severity of the clinical indication, and the clinical relevance of the test results, without having to read through many pages of a complex lab report which may be difficult to interpret. Therefore, such a navigator display may advantageously facilitate understanding and interpretation of genetic test results.

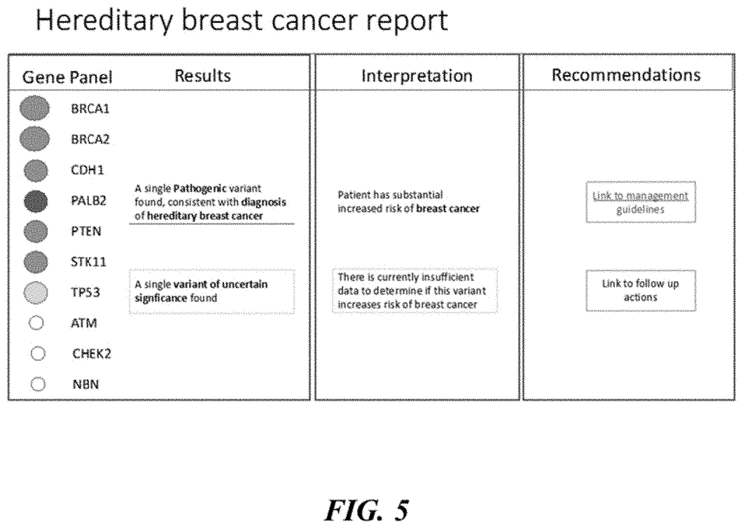

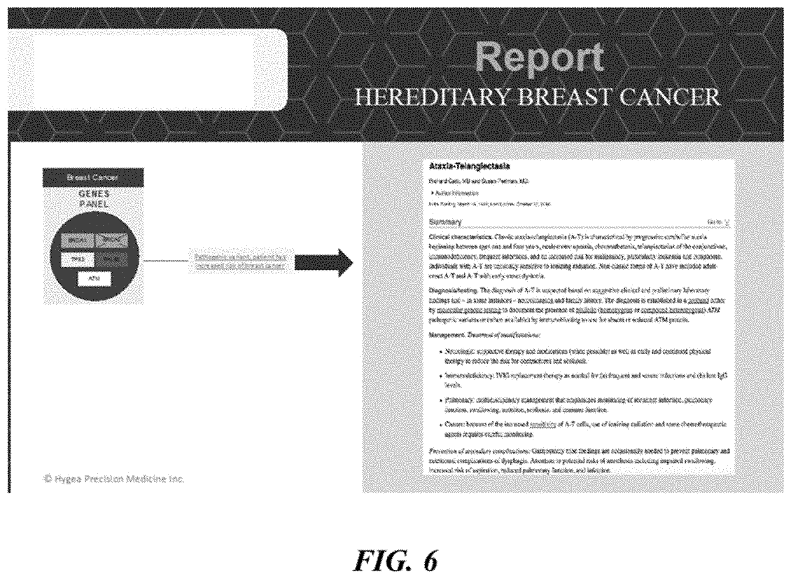

FIGS. 5-7 illustrate visuals that may help physicians to understand genetic tests and test results and to make clinical decisions based on the test results. For example, FIG. 5 illustrates an example of a hereditary breast cancer report, which includes results of genetic analysis of a breast cancer gene panel with color coding, and reviewable events, in accordance with disclosed embodiments. As another example, FIG. 6 illustrates an example of a hereditary breast cancer report, which includes results of genetic analysis of a breast cancer gene panel with color coding, in accordance with disclosed embodiments.

For example, a gene may be colored green to indicate that no variations of that gene were detected in the genetic test of the subject. As another example, a gene may be colored red along with a displayed flag with a number that reflects the number of reviewable events for this gene. A reviewable event may indicate that a pathogenic variant may been found and that the physician should take a second look. A hyperlink may be provided to bring up management guidelines, and the complete lab report if needed. A special color coding may be used to indicate if a genetic variation is heterozygous or homozygous. A gene may be colored yellow to indicate that a single variant of uncertain significance has been found for the gene. A gene may be colored red to indicate that pathogenic variations have been found for the gene. In case the sequencing quality is sub-optimal, there may be a significant probability of a false positive or a false negative occurring. Such a significant probability may be indicated by a cross on the gene in the gene panel.

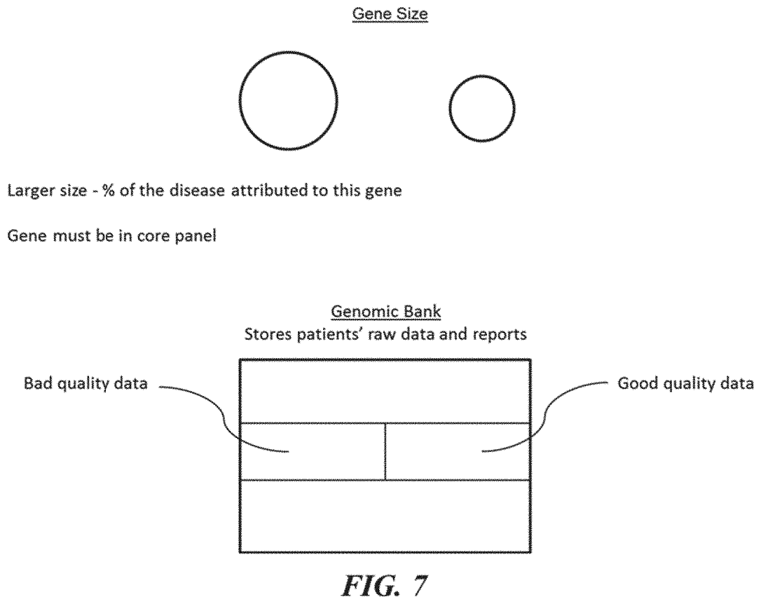

FIG. 7 illustrates an example of how gene size can be visualized to reflect probability and quality of data in the genome bank. The genes may be displayed as circles with different sizes, with the size of the gene indicating the probability that the disease attributes to this gene. In other words, the larger the size of a circle displayed to represent a given gene, the higher the probability that the disease can be attributed to this gene mutation.

The graphical genome system may analyze acquired information from a subject (patient) and a clinical indication determined by a physician of the subject to generate an output of genomic information of the subject having the clinical indication. For example, the system may apply a classification algorithm to the acquired information from the subject and the clinical indication (e.g., a clinical guidance) determined by the physician of the subject to generate the output of genomic information (e.g., a list of genes) of the subject having the clinical indication. The classification algorithm may comprise an artificial intelligence-based classifier, such as a machine learning based classifier, configured to process the acquired information from the subject and the clinical indication determined by the physician of the subject to generate the output of genomic information of the subject having the clinical indication. The machine learning classifier may be trained using datasets from one or more sets of subjects with a given clinical indication as inputs and known genomic information (e.g., genes which experience a significantly higher or significantly lower mutation rate as compared to a reference genome) of the subjects as outputs to the machine learning classifier.

The machine learning classifier may comprise one or more artificial intelligence-based algorithms. In some embodiments, the artificial intelligence-based algorithm comprises a machine learning algorithm. In some embodiments, the machine learning algorithm is selected from the group consisting of: a support vector machine (SVM), a Naïve Bayes classification, a random forest, and a neural network. Examples of machine learning algorithms may include a support vector machine (SVM), a naïve Bayes classification, a random forest, a neural network, deep learning, or other supervised learning algorithm or unsupervised learning algorithm for classification and regression. The machine learning classifier may be trained using one or more training datasets corresponding to subject data.

Training datasets may be generated from, for example, one or more sets of subjects having common characteristics (e.g., features such as clinical indications) and outcomes (e.g., labels such as known genomic information (e.g., genes which experience a significantly higher or significantly lower mutation rate as compared to a reference genome) of the subjects). Training datasets may comprise a set of features and labels corresponding to the features. Features may comprise characteristics such as, for example, categories of clinical indications, such as a disease or a disorder.

Labels may comprise outcomes such as, for example, genomic information (e.g., including gene mutations) of the subject having the clinical indication. Outcomes may include a characteristic associated with the clinical indication of the subject.

Training sets (e.g., training datasets) may be selected by random sampling of a set of data corresponding to one or more sets of subjects. Alternatively, training sets (e.g., training datasets) may be selected by proportionate sampling of a set of data corresponding to one or more sets of subjects. The machine learning classifier may be trained until certain predetermined conditions for accuracy or performance is satisfied, such as having minimum desired values corresponding to accuracy measures. For example, the accuracy measure may correspond to classification of known genomic information of a clinical indication in the subject.

The artificial intelligence algorithm may comprise an optimization based on a constraint comprising one or more of: a number of the one or more genetic tests, a cost (e.g., a total cost or a cost reimbursed by insurance) of the one or more genetic tests, a total number of genes assayed by (e.g., coverage of) or the quality of the one or more genetic tests, and a diagnostic metric (e.g., accuracy, confidence, sensitivity, specificity, positive predictive value, or negative predictive value) of the evaluation of the clinical indication. For example, the constraint on a number of genetic tests to be recommended may be no more than 1, no more than 2, no more than 3, no more than 4, no more than 5, no more than 6, no more than 7, no more than 8, no more than 9, or no more than 10 genetic tests. As another example, the constraint on a cost of genetic tests to be recommended may be no more than $100, no more than $200, no more than $500, no more than $1,000, no more than $2,000, no more than $5,000, no more than $10,000, no more than $20,000, no more than $50,000, or no more than $100,000 to perform all of the set of genetic tests.

As another example, the constraint on a diagnostic metric (e.g., accuracy, confidence, sensitivity, specificity, positive predictive value, or negative predictive value) of the evaluation of the clinical indication may be that the diagnostic metric of the evaluation of the clinical indication meet a predetermined threshold criterion. For example, the predetermined threshold criterion (e.g., corresponding to an accuracy, confidence, sensitivity, specificity, positive predictive value, or negative predictive value) may be at least about 50%, at least about 55%, at least about 60%, at least about 65%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%.

In some embodiments, the system further comprises allowing hospitals to track and analyze data (e.g., genomic health records) across a population of patients. In a further embodiment, hospitals track and analyze data for population profiling. In a further embodiment, hospitals track and analyze data for tracking of metrics such as total genetic test spend for a population of patients.

In some embodiments, the system allows hospitals to track and analyze information with respect to genetic tests. In a further embodiment, hospitals track and analyze information as to who is requesting genomic health data, what genomic health data is being requested, when genomic health data is being requested, or where the genomic health data is being requested from.

In some embodiments, the system allows hospitals to track and analyze information with respect to a patient's feedback. In a further embodiment, hospitals track and analyze information as to whether patients are receiving the correct therapeutic regime for their diagnosis. In a further embodiment, hospitals track and analyze information as to whether recommendations by physicians are being followed (e.g., recommended genetic tests, prescribed therapeutics, or diet, behavioral, or other lifestyle changes or habits). In a further embodiment, hospitals track and analyze information in order to correlate health outcomes and side effects.

In some embodiments, the system allows hospitals to track and analyze information with respect to population profiling. In a further embodiment, hospitals track and analyze information about undiagnosed patients. In a further embodiment, hospitals track and analyze data correlating genetic variants with various diseases (e.g., correlating patient's diseases or disorders with their genomic health data and other health information). In a further embodiment, hospitals track and analyze data to determine the number of patients being diagnosed with various diseases (e.g., correlating patient diagnoses with their genomic health data and other health information). In a further embodiment, hospitals track and analyze data to determine the number of patients being diagnosed with a rare disease.

In some embodiments, the system allows hospitals to track and analyze information with respect to the quality of the genomic health data. In a further embodiment, hospitals track and analyze how often patients receive their genomic health data. In a further embodiment, hospitals track and analyze whether genetic risks are being documented in the EHR. In a further embodiment, hospitals track and analyze whether patients are avoiding exposures in line with their genomic health data e.g., by following physician recommendations regarding diet, behavioral, or other lifestyle changes or habits).

In one aspect, the present disclosure provides a system that would allow for an increase of genetic tests being ordered by operators and patients. In some embodiments, this may results in an increased of genetic tests ordered by CLIA-certified laboratories.

In another aspect, the present disclosure provides a system where patients upload genetic or genomic sequencing data. In some embodiments, patients use the system to store sequencing data. In some embodiments, patients use the system to access genetic or genomic sequencing data at any location or at any time. In some embodiments, patients use the system to share data with other individuals. In a further embodiment, patients use the system to share data with healthcare professionals. In a further embodiment, patients use the system to share data with family members. In some embodiments, patients use the system to securely store genetic data e.g., using a secure cloud server, encrypted communications, encrypted storage systems, password-protected system access, access logs, or a combination thereof). In a further embodiment, patients may be granted a commitment that the system will not allow access to third parties. In some embodiments, patients use the system to connect with other patients with similar diseases. This may provide support and guidance to these patients. In some embodiments, patients pay a membership fee to use the system or to grant others access to the system.

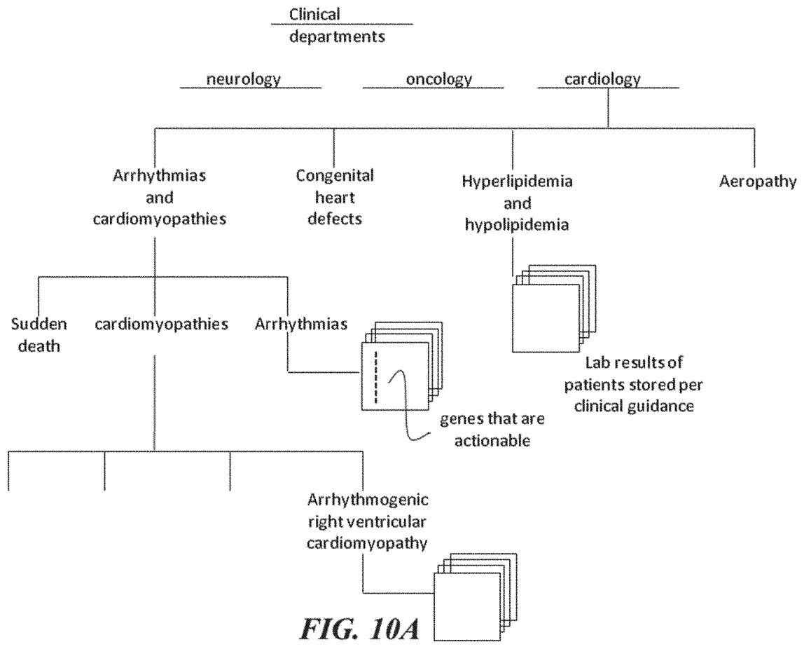

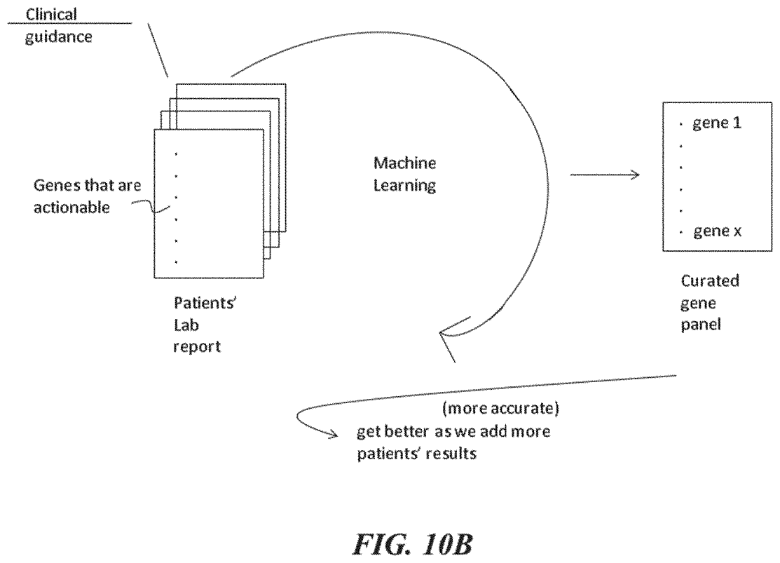

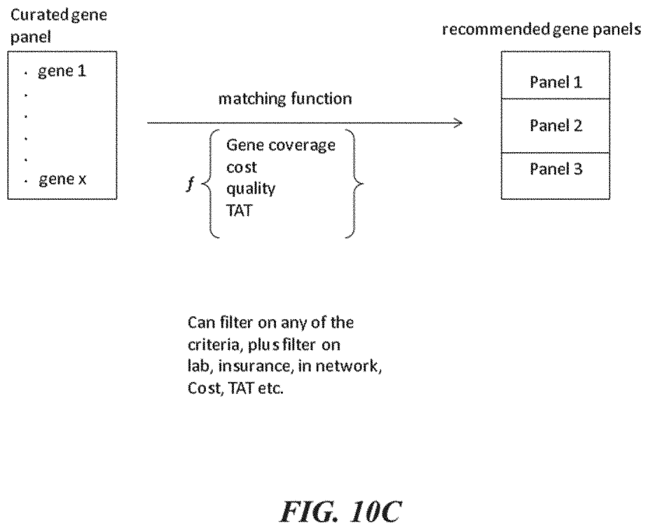

In another aspect disclosed herein, the graphical genomic system comprises machine learning-based gene panel selection that is customized to a hospital population. For example, FIG. 10A-10C illustrates an example of a navigator diagnostic workflow, in accordance with disclosed embodiments. The navigator may be a context sensitive user interface that responds in real time.

A curated gene panel may be from open source or literature. This curated gene panel may have gene mutations that come from various demographics. The curated genes may be different by demographic and if customized to the hospital patient population, it may be more accurate and yield better care for the patients. For example younger population may have more early onset gene panels.

The navigator may include a clinical view (see FIG. 10A). The clinical view may guide the physician based on the clinical guidance he or she provides. For example, the clinical view may display a number of different clinical departments, such as: Cardiology, Immunology, Oncology, Neurology, Dermatology, etc. Suppose that the physician chooses Cardiology as the clinical department in the clinical view. Then the navigator may generate a disease view that displays a number of different disease categories, such as: Arrhythmias and Cardiomyopathies, Congenital heart defects, Hyperlipidemia and hypolipidemia, Aeropathy, etc.

Suppose the physician chooses Neurology as the clinical department in the clinical view. Then the navigator may generate a disease view that displays a number of different categories, such as: Dementia, Movement disorder, White Matter disease, Epilepsy, Fatigue syndrome, etc.

Suppose the physician chooses Oncology as the clinical department in the clinical view. Then the navigator may generate a disease view that displays a number of different categories, such as: Lung, Colon, Bladder, Beast, Ovarian, Melanoma, etc.

Under each of these disease categories, the lab results of the patient may be stored for clinical guidance. Suppose the physician selects a certain disease categories, further disease subcategories may be displayed and in each of these disease subcategories, panels may be shown. For example, if the physician selects the disease category of Arrhythmias and cardiomyopathies, then further disease categories may be displayed such as: Sudden death, Cardiomyopathies, Arrhythmias, etc. Suppose the physician were to select one of these disease subcategories, a panel may be displayed that shows genes that may be actionable.

Suppose the clinical guidance for the physician provides genes that are actionable from a patients' lab report, machine learning may provide a curated gene panel (see FIG. 10B). This curated gene panel may improve in accuracy as more patients' results are added. From the curated gene panel, a number of parameters may be used to filter the panels. These parameters may be gene coverage, the cost of the test, the quality of the data, turnaround time (TAT), insurance (in-network, out-of-network), deletion/duplication, variation coverage, etc.

Where a gene panel is filtered based on these parameters, a series of recommended gene panels may be displayed (see FIG. 10C). At least one recommended gene panel may be displayed.

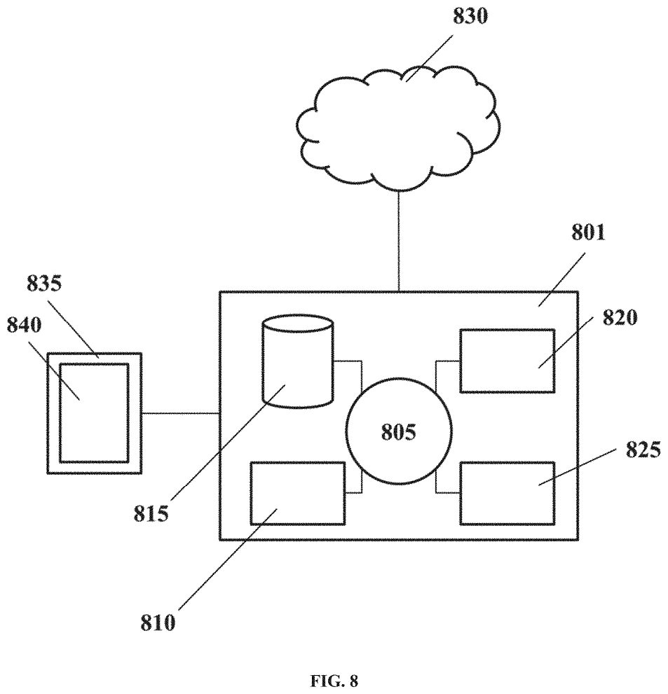

The present disclosure provides computer systems that are programmed to implement methods of the disclosure. FIG. 8 shows a computer system 801 that is programmed or otherwise configured to, for example, (i) receive a request from a computer of an operator to evaluate a clinical indication of a subject, (ii) upon receiving the request, determine genomic data relevant to the clinical indication, and (iii) generate an output comprising a recommendation that the operator have the genomic data obtained for the subject.

The computer system 801 can regulate various aspects of analysis, calculation, and generation of the present disclosure, such as, for example, (i) receiving a request from a computer of an operator to evaluate a clinical indication of a subject, (ii) upon receiving the request, determining genomic data relevant to the clinical indication, and (iii) generating an output comprising a recommendation that the operator have the genomic data obtained for the subject. The computer system 801 can be an electronic device of a user or a computer system that is remotely located with respect to the electronic device. The electronic device can be a mobile electronic device.

The computer system 801 includes a central processing unit (CPU, also “processor” and “computer processor” herein) 805, which can be a single core or multi core processor, or a plurality of processors for parallel processing. The computer system 801 also includes memory or memory location 810 (e.g., random-access memory, read-only memory, flash memory), electronic storage unit 815 (e.g., hard disk), communication interface 820 (e.g., network adapter) for communicating with one or more other systems, and peripheral devices 825, such as cache, other memory, data storage and/or electronic display adapters. The memory 810, storage unit 815, interface 820 and peripheral devices 825 are in communication with the CPU 805 through a communication bus (solid lines), such as a motherboard. The storage unit 815 can be a data storage unit (or data repository) for storing data. The computer system 801 can be operatively coupled to a computer network (“network”) 850 with the aid of the communication interface 820. The network 830 can be the Internet, an internet and/or extranet, or an intranet and/or extranet that is in communication with the Internet.

The network 830 in some cases is a telecommunication and/or data network. The network 830 can include one or more computer servers, which can enable distributed computing, such as cloud computing. For example, one or more computer servers may enable cloud computing over the network 830 (“the cloud”) to perform various aspects of analysis, calculation, and generation of the present disclosure, such as, for example, (i) receiving a request from a computer of an operator to evaluate a clinical indication of a subject, (ii) upon receiving the request, determining genomic data relevant to the clinical indication, and (iii) generating an output comprising a recommendation that the operator have the genomic data obtained for the subject. Such cloud computing may be provided by cloud computing platforms such as, for example, Amazon Web Services (AWS), Microsoft Azure, Google Cloud Platform, and IBM cloud. The network 830, in some cases with the aid of the computer system 801, can implement a peer-to-peer network, which may enable devices coupled to the computer system 801 to behave as a client or a server.

The CPU 805 may comprise one or more computer processors and/or one or more graphics processing units (GPUs). The CPU 805 can execute a sequence of machine-readable instructions, which can be embodied in a program or software. The instructions may be stored in a memory location, such as the memory 810. The instructions can be directed to the CPU 805, which can subsequently program or otherwise configure the CPU 805 to implement methods of the present disclosure. Examples of operations performed by the CPU 805 can include fetch, decode, execute, and writeback.

The CPU 805 can be part of a circuit, such as an integrated circuit. One or more other components of the system 801 can be included in the circuit. In some cases, the circuit is an application specific integrated circuit (ASIC).

The storage unit 815 can store files, such as drivers, libraries and saved programs. The storage unit 815 can store user data, e.g., user preferences and user programs. The computer system 801 in some cases can include one or more additional data storage units that are external to the computer system 801, such as located on a remote server that is in communication with the computer system 801 through an intranet or the Internet.

The computer system 801 can communicate with one or more remote computer systems through the network 830. For instance, the computer system 801 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 801 via the network 830.

Methods as described herein can be implemented by way of machine (e.g., computer processor) executable code stored on an electronic storage location of the computer system 801, such as, for example, on the memory 810 or electronic storage unit 815. The machine executable or machine readable code can be provided in the form of software. During use, the code can be executed by the processor 805. In some cases, the code can be retrieved from the storage unit 815 and stored on the memory 810 for ready access by the processor 805. In some situations, the electronic storage unit 815 can be precluded, and machine-executable instructions are stored on memory 810.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 801, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 801 can include or be in communication with an electronic display 835 that comprises a user interface (UI) 840 for providing, for example, a visual display indicative of a clinical view, a disease category view, a disease view, and/or a genes view. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 805. The algorithm can, for example, (i) receive a request from a computer of an operator to evaluate a clinical indication of a subject, (ii) upon receiving the request, determine genomic data relevant to the clinical indication, and (iii) generate an output comprising a recommendation that the operator have the genomic data obtained for the subject.

### EXAMPLES

### Example 1

A 25-year-old patient presents with symptoms of chest pains, which occur especially when exercising. The patient's father had a heart attack in his 50s. The patient's physician is aware that having heart problems at such a young age may be indicative of a hereditary disease or disorder. He refers the patient for basic clinical testing first (EKG, Echocardiogram, etc.). Laboratory reports provide a clinical indication of Hypertrophic cardiomyopathy. The physician is aware that this disease may be hereditary but is not sure which genetic tests to order for the patient. There are about 600 different genetic tests commercially available for cardiomyopathy in the United States, offered by about 40 different laboratories. The physician decides to order the core cardiology panel which he is familiar with. Laboratory results for this core cardiology panel come back negative, and no further clinical action is taken for the patient. The physician is not aware that there are extended gene panels available which he can order for the patient.

Using methods and systems of the present disclosure, the physician runs the navigator and provides the patient's information, clinical history, and Hypertrophic cardiomyopathy as a clinical guidance. The navigator identifies a core gene panel and an extended gene panel in response to the physician's input data, and provides a recommendation for the physician to order the extended gene panel for the patient. The physician orders the extended gene panel through the navigator, and three days later, receives a notification on his computer that the genetic test results are ready. The navigator presents a visual view of the genetic test results. Every analyzed gene is shown as a circle, each of which is green except for one gene, which is colored red. This gene has a pathogenic variant. The genetic test results reveal that the patient has inherited cardiomyopathy, and the physician was able to perform an accurate diagnosis and provide customize care for the patient, using the navigator.

### Example 2

A 10-year-old girl, who is otherwise healthy, presents with 5 unprovoked epileptic seizures in the past year. Brain imaging of the patient does not show a clear cause of the seizures, and genetic testing is recommended. The patient's neurologist orders an 87-gene seizure panel to be performed. Two genetic changes are reported based on the seizure panel, but they do not provide a satisfactory diagnosis. The neurologist requests whole exome sequencing of the patient's DNA sample and of the patient's parents' DNA samples. In the meantime, the patient is referred to a geneticist. The geneticist receives only a grainy scan of the test report. One variant, a deletion in CLN3, is difficult to interpret clinically because the report does not detail the size of the deletion and the geneticist has no access to the raw data. Meanwhile, the exome sequencing test is performed, but the results again are not accessible to the geneticist except by fax through the neurologist.

Using methods and systems of the present disclosure, the geneticist is easily able to access the patient's test report and raw data, and can see the precise genetic testing results, even those not reported clearly by the lab. It is clearly communicated to the neurologist that the patient's DNA does not contain a second variant and that CLN3 is likely not a diagnosis, because the navigator has an improved interface that clearly indicates in a graphical manner which gene variants are found and which gene variants are not found. When new exome results are available, they are easily transmitted to the geneticist, who can review them. Using the navigator, both the patient and the neurologist can send the results to the geneticist.

### Example 3

A 15-year-old boy presents with symptoms of progressive ataxia for the past year without a definitive clinical diagnosis. The patient received a brain MRI with abnormal results, indicating an injury in the brain. The patient's family recalls having genetic testing started, but is unsure if such genetic testing was completed, when it was performed, or who received the results. The geneticist who ordered the genetic tests had recently moved to another institution. After making several phone calls and submitting forms over more than a day, it was determined that the genetic results had been available for 3 weeks and that a genetic diagnosis had been made. In the meantime, the patient has received several unnecessary and risky therapies and was literally minutes away from being exposed to a high-dose radiation therapy for another, unnecessary, imaging study.

Using methods and systems of the present disclosure, the patient is continuously made aware of genetic test results. The patient and his physicians are automatically alerted when genetic test results become available, since the patient's family, having previously been granted access, can give approval to provide a navigator user account (with login and password), allowing immediate access of the patient's genetic testing history to any clinical provider.

### Example 4

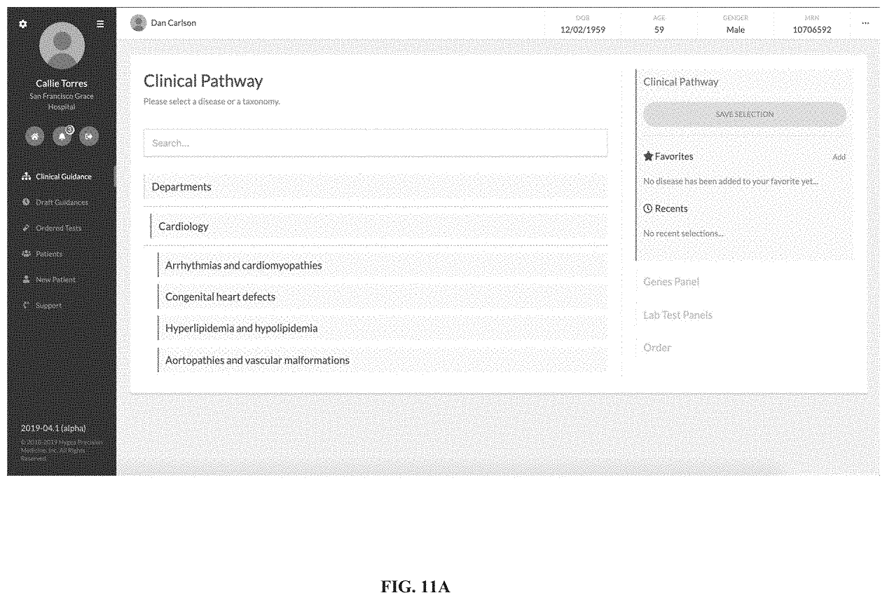

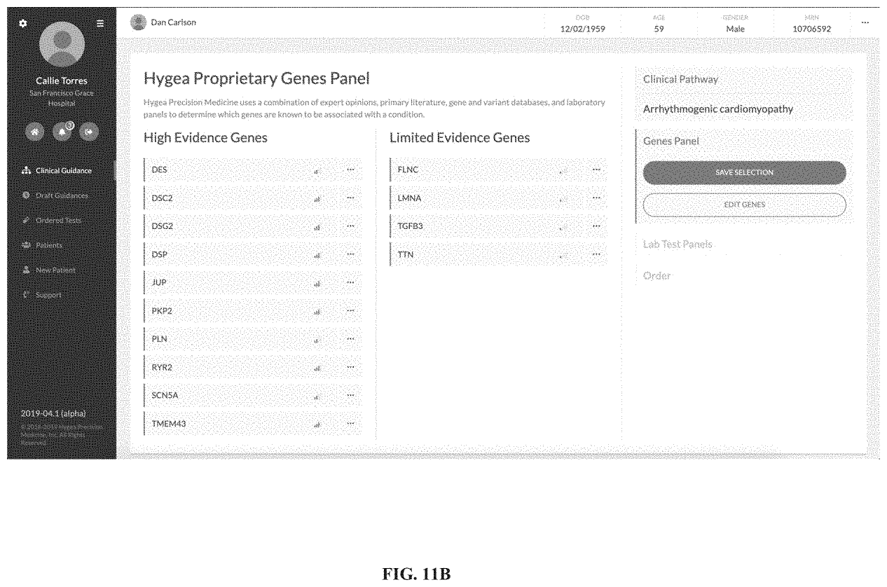

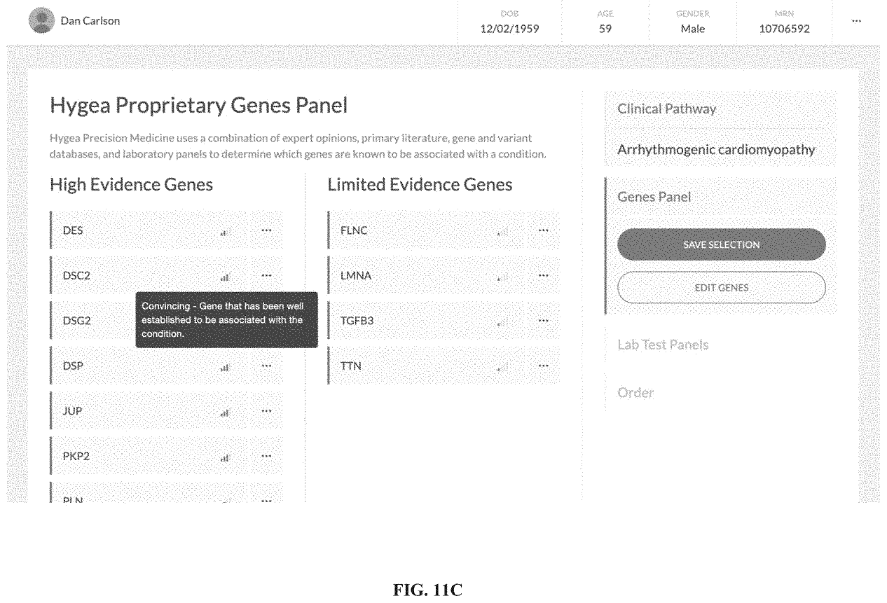

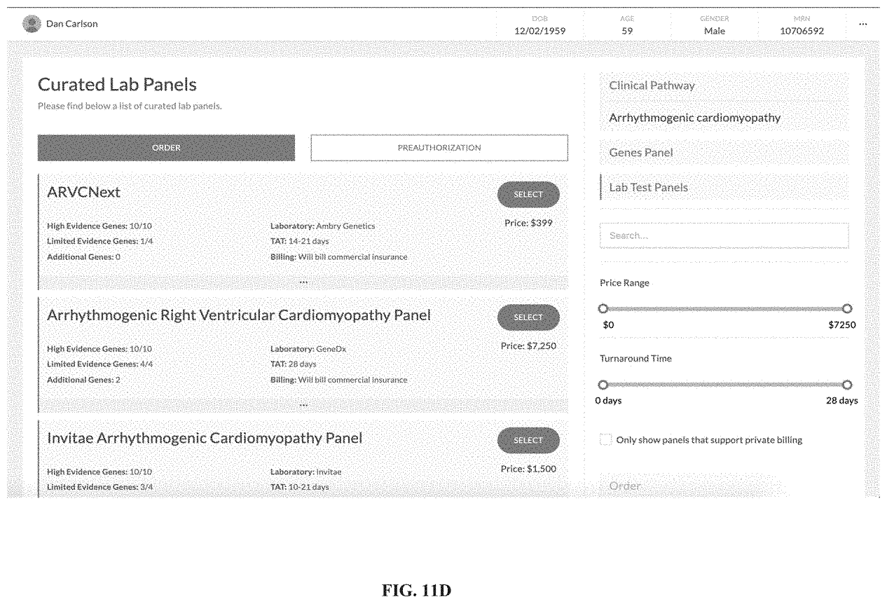

FIGS. 11A-11D illustrate examples of a navigator software of a graphical genome system for use by clinical providers, including a view of a list of clinical pathways (FIG. 11A), views of a proprietary genes panel (FIGS. 11B-11C), and a view of a list of curated lab panels (FIG. 11D). For example, the clinical provider can use the view of a list of clinical pathways (FIG. 11A) to select a disease or a taxonomy. In this example, the clinical provider chooses the Department of Cardiology, which causes the Navigator software to display a list of cardiology-related clinical pathways, including arrhythmias and cardiomyopathies, congenital heart defects, hyperlipidemia and hypolipidemia, and aortopathies and vascular malformations.

The clinical provider can select a given clinical pathway from the list of clinical pathways, which causes the Navigator software to display a proprietary genes panel (FIGS. 11B-11C). The proprietary genes panel is generated based on analysis of a combination of expert opinions, primary literature, gene and variant databases, and laboratory panels to determine which genes are known to be associated with a condition. For example, the proprietary genes panel can include a list of high-evidence genes (or convincing-evidence) genes, probable-evidence genes, and limited-evidence genes. High-evidence (or convincing-evidence) genes refer to those genes that have a well-established association with a condition. Probable-evidence genes refer to those genes with reasonable evidence of an association with a condition. Limited-evidence genes have limited, newer, and/or conflicting evidence with respect to an association with a condition. The proprietary genes panel can indicate high-evidence genes (or convincing-evidence) genes, probable-evidence genes, and limited-evidence genes visually using different color codes and/or different symbols.

The clinical provider can select one or more genes for further patient testing based on their description and/or categorization as high-evidence genes (or convincing-evidence) genes, probable-evidence genes, or limited-evidence genes, which causes the Navigator software to display a list of curated lab panels (FIG. 11D). These curated lab panels are a collection of gene panels which are commercially available for genetic testing from a large set of certified labs (e.g., about 200 different certified labs) at different prices. Using the Navigator, the clinical provider can order the most efficient and accurate gene panels and perform an optimal selection of a set of one or more genetic tests to be performed for the patient, given particular constraints (e.g., lowest price, highest sensitivity of diagnosing a condition, highest specificity of diagnosing a condition, highest accuracy of diagnosing a condition, highest reimbursement allowance, etc.). For example, an arrhythmogenic right ventricular cardiomyopathy panel can contain 10 out of 10 high-evidence genes and 4 out of 4 limited-evidence genes, which is available at a price of $7,250 with a turnaround time (TAT) of 28 days and the ability to automatically handle the billing of commercial insurance. Using the Navigator, the clinical provider can automatically order the required genetic tests electronically, without the need to manually fill out and submit different forms for each individual gene panel or genetic test ordered. Optionally, the clinical provider can view a user-selected subset of lab panels, such as only panels that support private billing, only panels that fall within a selected price range, only panels that fall within a selected range of turnaround time, or a combination thereof.

### Example 5

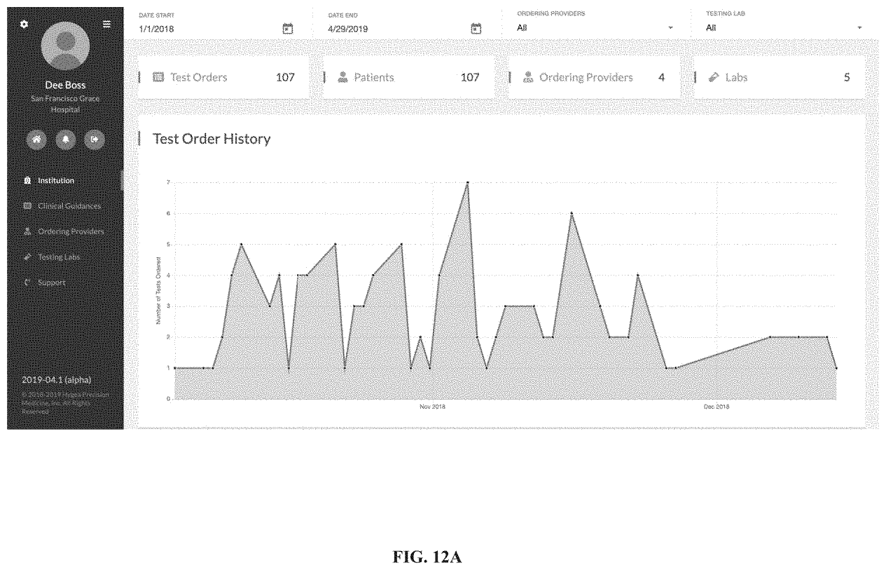

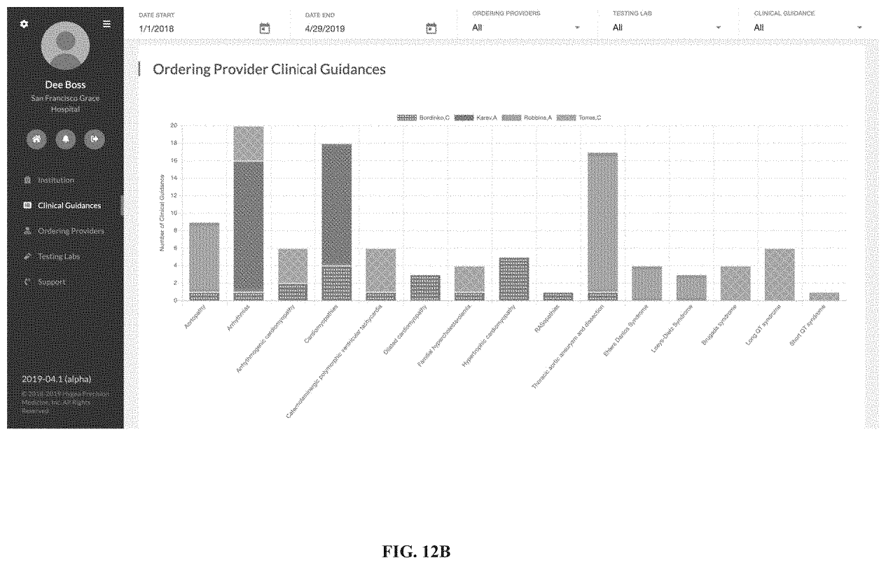

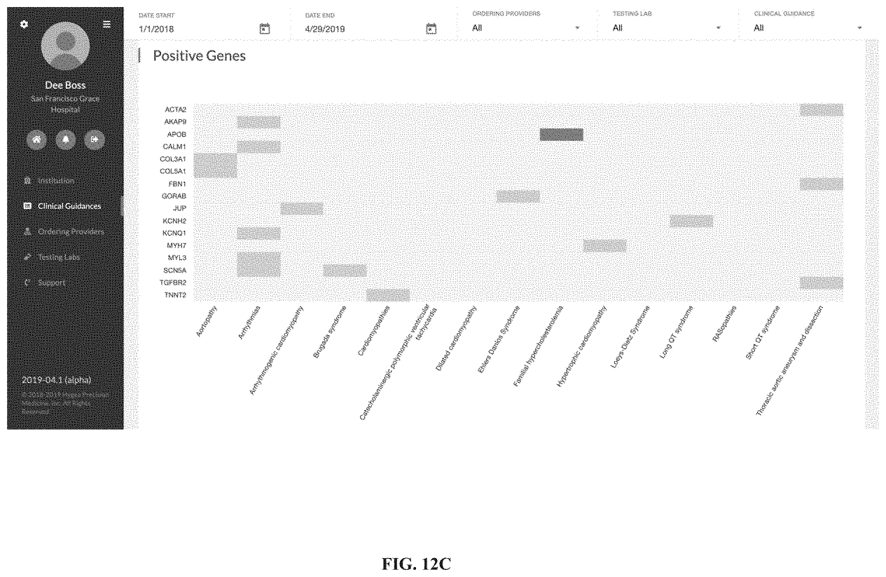

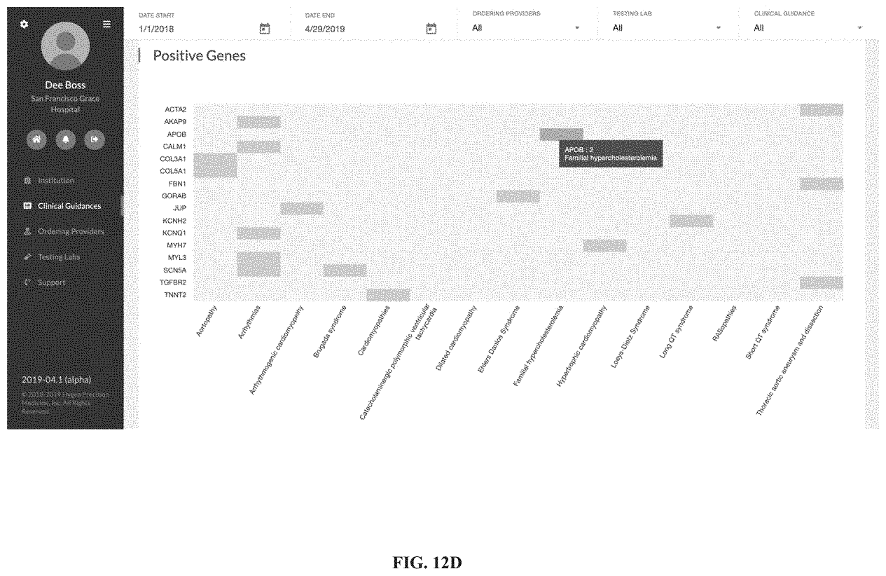

FIGS. 12A-12D illustrate examples of analytics tools of a navigator software of a graphical genome system for use by ordering clinical providers, including a view of a test order history (FIG. 12A), a view of clinical guidances for different diseases or disorders (FIG. 12B), and views of a list of positive genes (FIGS. 12C-12D). The view of the test order history displays a plot of the number of tests ordered by the clinical provider over time (e.g., across different months) (FIG. 12A). The view of the clinical guidances for different diseases or disorders shows the number of clinical guidances selected or indicated by the clinical provider over a period of time (FIG. 12B). In this example, the list of clinical guidances includes aortophathy, arrhythmias, arrhythmogenic cardiomyopathy, cardiomyopathies, catecholaminergic polymorphic ventricular tachycardia, dilated cardiomyopathy, familial hypercholesterolemia, hypertrophic cardiomyopathy, RASopathies, thoracic aortic aneurysm and dissection, Ehlers Danlos Syndrome, Loeys-Dietz Syndrome, Brugada syndrome, long QT syndrome, and short QT syndrome; with numbers of clinical guidances ranging from 1 to 20 during the time period of Jan. 1, 2018 to Apr. 29, 2019 across all ordering providers and testing labs. The views of lists of positive genes shows positive genes that are positively associated with different conditions (e.g., diseases or disorders) using shaded boxes (FIGS. 12C-12D).

In this example, the positive gene associations include COL3A1 and COL5A1 genes for aortopathy; AKAP9, CALM1, KCNQ1, MYL3, and SCN5A genes for arrhythmias; JUP gene for arrhythmogenic cardiomyopathy; SCN5A gene for Brugada syndrome; TNNT2 gene for cardiomyopathies; no genes for catecholaminergic polymorphic ventricular tachycardia; no genes for dilated cardiomyopathy; GORAB gene for Ehlers Danlos Syndrome; APOB gene for familial hypercholesterolemia (with a strong positive association); MYH7 gene for hypertrophic cardiomyopathy; no genes for Loeys-Dietz Syndrome; KCNH2 gene for long QT syndrome; no genes for RASopathies; no genes for short QT syndrome; ACTA2, FBN1, and TGFBR2 genes for thoracic aortic aneurysm and dissection; etc.

### Example 6

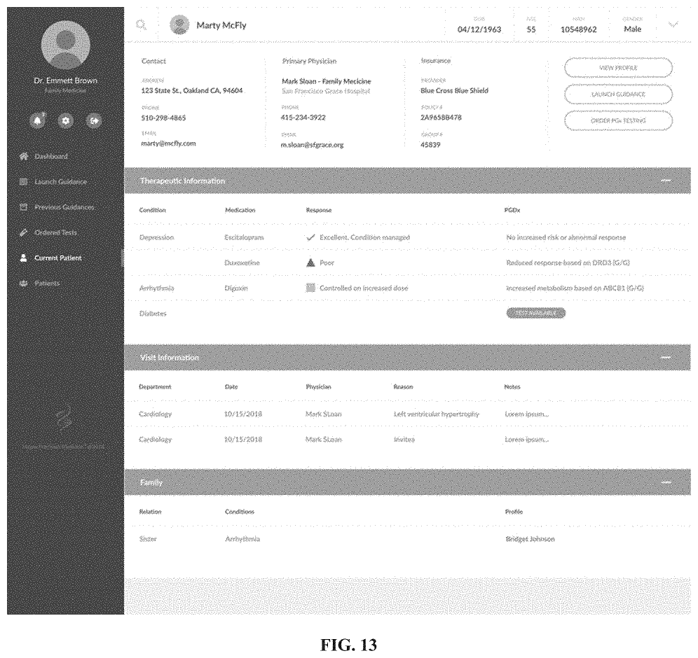

Using systems and methods of the present disclosure, pharmacogenomic data can be integrated into patients' clinical data for further analysis. Pharmacogenomic data generally refers to data related to the role of the genome in drug response, which is related to analysis of how the genetic makeup of an individual affects his or her response to drugs. FIG. 13 illustrates an example of the design and functionality of a Navigator software of a graphical genome system for use with pharmacogenomic data. In this design, a patient's medical conditions (e.g., diseases and/or disorders) are listed on the far left, which are organized by date of diagnosis (with the most recent being at the top). Medications (e.g., prescription drugs) are listed by medical condition and are linked to the medical conditions. If there are multiple medications per medical condition, the first medication(s) listed are the most recently prescribed and medications the patient is actively taking. Medications are color-coded based on the pharmacogenomic data (e.g., a green check indicating an excellent response such that a condition is managed, a yellow square indicating an intermediate response such that a condition is controlled on increased dosage, or a red “X” indicating a poor response). Additionally, when pharmacogenomic testing may be beneficial for a given medical condition or medication of the patient, then a notification “Test available” is displayed.

Pharmacogenomic data can be linked to a patient's health record based on the medical conditions and medications present in the patient's health record. Generally, pharmacogenomic data is most informative when it is integrated with the medications (and potential conditions) that are associated with a given patient. Therefore, a database is maintained with the medications for which pharmacogenomic data is most beneficial, and the medications are linked to the medical conditions for which they are commonly prescribed. This allows the software to note when pharmacogenomic testing should be considered based on the patient's condition prior to a medication being prescribed.

As an example of a user story, suppose Dr. Smith, a clinical provider, is finishing up seeing his last patient for the day when he receives a notification from the Navigator software. Janelle, Dr. Smith's patient for whom he ordered a broad pharmacogenetics (PGx) panel a year ago, was just prescribed a medication by a gastroenterologist. This medication was noted with a red “X” based on the PGx data. Dr. Smith checks the Navigator for a more suitable medication and, in consultation with the gastroenterologist, changes the medication prior to the prescription being filled at the pharmacy.

As another example of a user story, suppose Dr. Adams, another clinical provider, receives concerning blood work results back on her patient, Tim. She logs into Tim's chart in the Navigator software and updates his clinical health record information to note that Tim is now diagnosed with diabetes, based on the blood work results and his symptoms. Tim had previously chosen to have PGx testing several years earlier. Had he not had this testing, the Navigator software would have alerted Dr. Adams that PGx testing may be beneficial before prescribing any medication. As Dr. Adams looks for a suitable medication for Tim, she notices that each medication is accompanied by a color-coded indicator showing as green, yellow, or red, based on Tim's prior PGx results, and therefore is able to quickly and efficiently choose the best medication to prescribe for Tim, based on his condition and prior PGx testing.

Generally, in order for the PGx portion of the Navigator to work optimally, there may be several key features that may need to be added. First, clinical health records for all of the physician's patients should be stored in the Navigator, or at least all those with PGx data. Patients' records can be imported into the Navigator at that time of creating a new clinical guidance. Second, patient data needs to be updated regularly or in real time with the integrated EMR system. This can be performed by the Navigator, which updates all patient data, assesses the new data (e.g., a new medication or condition) to determine if there are issues or concerns as it relates to PGx, and generates a color-coded notification to the provider indicating the issue. Third, the Navigator may handle or facilitate the prescribing process in order to make this easier for a provider. For example, the Navigator may have built-in functionality to allow prescribing of medications or may be integrated with the prescribing system of the clinical provider. Through this approach, the provider avoids the need to log into the Navigator, review which medications are potential issues for the patient given his or her PGx data, identify an appropriate medication for the patient's condition, and then log into their prescribing software and find and choose the same medication.

### Example 7

A new patient is being seen in the office of cardiologist Dr. Sloan. Rosalie Estrada was referred due to arrhythmia and an abnormality of right ventricle. Dr. Sloan discusses management of her condition. Dr. Sloan believes that her symptoms (syncope, ventricular tachycardia, and right ventricular dyskinesia) as well as her family history (cardiomyopathy) are consistent with a condition known as ARVC (arrhythmogenic right ventricular cardiomyopathy). Dr. Sloan is aware that there are several genes known to cause ARVC and that there are multiple panels available to test for ARVC. However, he does not know which genes should be included in such a test, he is not certain which are the best options for an ARVC genetic panel, and he is not sure of the typical price range for such a panel. The provider and the patient discuss options for ordering genetic testing. The patient is interested in genetic testing, but notes that she is concerned about the cost.

Using systems and methods of the present disclosure, Dr. Sloan uses the Navigator to aid in selecting and ordering genetic tests. The Navigator supports the ability to enroll multiple providers from the same institution and practice. First, Dr. Sloan logs into the Navigator using his username and password.

Once logged in, the provider views his dashboard. The dashboard provides the provider with information about his patients divided up based on the status of their genetic testing. On the left sidebar, there are three circles, notifications that provide messages about the status of testing, settings, and log out. There are also five links: dashboard (to bring the provider back to the dashboard when they are in another page), clinical guidance (to start a guidance), draft guidances (to return to a guidance that was incomplete), ordered tests (a list of all pending and completed genetic tests ordered), and patients (a list of all patients). The dashboard also provides a list of the most recent completed tests, a list of all pending tests, and a search bar to look up any patient in the Navigator.

The completed tests section lists out any patients who have had a recently completed genetic test organized by the most recently completed. On his dashboard, he also has a list of pending tests and is able to quickly see that he has 3 genetic tests pending and when they were ordered. On the dashboard, the provider scrolls down to the pending tests section.

These two lists of pending tests can be filtered. The provider remembers that he recently received results back from a certain laboratory but does not remember the name of the patient. He types in the name of the laboratory into the filter field next to the completed tests to show only tests ordered from that laboratory.

These lists can also be organized by each of the categories listed (e.g. patient name, test name). The provider can click on “Name” to reorganized the list in alphabetical order.

When Dr. Sloan is consulting with his patient, Rosalie Estrada, he types in “Est” into the search bar, and clicks on the patient name “Rosalie Estrada” to pull up her patient record. The provider clicks the down arrow to the far right of the patient details to show more information about her history.

Within the Navigator, all of the patient's personal and family history are already noted. These items in an integrated system can pull from the providers EMR and in the stand alone version can be entered by the provider or staff. The patient's clinical information and family history allow for quick reference in choosing the most appropriate genetic test.

The Navigator supports additional features including Pharmacogenomics, ICD-10 linked to the clinical and family history, and the addition of other providers in this patient's care team for ease of sharing results.

After a brief discussion of genetic testing, the patient is interested and consents to have a genetic testing for ARVC but notes that she is concerned about the cost. The provider clicks the arrow in the upper right again to hide the details of the patient, then clicks on clinical guidance to begin a guidance for the patient. Dr. Sloan notes that on the right panel beside the clinical guidance is a place for any favorite clinical guidance (e.g., any that he uses often) and also a list of recent clinical guidances that he has chosen. ARVC can be located by clicking: Cardiology >Arrhythmias and Cardiomyopathies >Cardiomyopathies >Arrhythmogenic Right Ventricular Cardiomyopathy. Of note, as this condition is considered both an arrhythmia and a cardiomyopathy, it is also found in the arrhythmias category.

The provider can also search by the condition name and ICD-10 codes with a one to one match to the condition. Above cardiology is the ontology search bar, where the provider types in “i42” to display dilated cardiomyopathy and restrictive cardiomyopathy. The provider can delete “i42” and type in “rig” to show that ARVC can also be found by searching the condition name. The provider click on ARVC to choose that condition, clicks the three dots for ARVC to show a brief summary about the condition, and clicks save selection. The Navigator can also support searching by common acronyms.

The provider is able to click through a hierarchy of available options, categorized by department to find the patient's condition, ARVC. Once he captures this condition, the navigator displays a curated list of genes that are associated with the condition. The curated gene panel is based on a combination of expert opinion (such as ClinGen and Genomics England PanelApp), primary literature, gene and variant databases, and laboratory panels to determine the strength of association between genes and the condition. The Navigator software uses an algorithm to match this curated list against all possible genetic panels to provide a list of the closest matches to the curated list.

The provider notes that he is able to view a curated list of genes that is associated with the condition classified as high evidence and limited evidence genes. The provider can find out more information about the gene by clicking on the three dots. The provider can click on the three dots for RYR2, then click on the OMIM link to bring up the OMIM page for this gene. The Navigator can also support adding, deleting, and editing genes not listed as high or limited evidence to a curated list to create a custom panel for that provider.

The Navigator displays a list of available genetic tests that matches best with the gene list, maximizing the number of high evidence genes included while minimizing the number of additional genes (those not listed as high or limited evidence). The list of available tests is easily comparable based on price, turn around time, methodology, and the genes included. The provider clicks on “read more” for ARVCNext in order to see the list of genes included on that panel. Note that all of the genes included on the panel are display sorted by high evidence, limited evidence, or additional genes. The provider drops the right side price filter from $7250 to $1999 to remove the highest priced panels. Providers may also search for a panel name or laboratory. The provider types “Ambry” to show that only the panel from Ambry Genetics is displayed. The Navigator can support additional filters to modify the algorithm (e.g. incorporating limited evidence in the algorithm, only showing panels with all high evidence genes, etc.).

The provider notes that the ARVCNext panel has all of the noted high evidence genes and has the least expensive price. The patient consents to this test. Once a panel is selected, an order form is populated based on the information in the Navigator and the order can be sent to the selected laboratory electronically through the Navigator. The provider selects ARVCNext from Ambry Genetics. A test order form for the laboratory is now displayed with information pulled from the patient record. The Navigator can support populating clinical history and family history with ICD-10 codes. The Navigator can also support providing detailed instructions for the provider for the chosen test/laboratory.

When the testing is completed, the results are sent back through the Navigator, then stored in the software. The provider returns to the dashboard by clicking dashboard. One of his patients, Willie Reeves, recently had testing for familial hypercholesterolemia. The doctor logs into the Navigator and clicks on the view results tab to see the PDF of the result. He notes the patient has a positive result. The Navigator can support notification features that sends a message to the provider once the results are ready, sharing these results with other providers and to the patient, and flagging important results to quickly identify at a future time.

The Navigator also displays a list of clinical guidance that were run for patient but for which testing was not ordered. The Navigator can support returning to the point in the guidance where the user previously left off. The Navigator also allows for viewing all ordered tests (pending and completed) through the “Ordered Test” link on the left sidebar as well as viewing all patients from the Patients link on the left sidebar.

The provider can use analytics tools of the Navigator as follows. First, the provider logs into the system using his username and password. The analytics section provides statistical information organized by categories: institution, clinical guidance, physician, and testing lab.

Under institution, the user wishes to view how many tests were ordered in the month of November. The user changes the order date to list Nov. 1, 2018 to Nov. 30, 2018. The user can see 46 orders were placed in that month. The user can also subdivide these by lab or by provider. The user scrolls down to view the number of tests ordered by each doctor and the average cost of each test by doctor.

Under clinical guidance, the user wishes to view how many patients with thoracic aortic aneurysms are having genetic testing. The user filters the list for “Thoracic Aortic Aneurysm and Dissection.” The user notes that since 2018, 17 patients have this clinical guidance and have been tested, and that the majority were ordered by one provider. The user remove the filters, and all of the clinical guidances return to view.

The user looks at the chart and can see one provider, Dr. Karev, is ordering testing under the more generic clinical guidance terms arrhythmias and cardiomyopathies which will likely lead to large, high cost panels being routinely ordered. The user scrolls down to see positive genes by clinical guidance and the clinical guidance per laboratory.

The user returns to institution, and scrolls down to view the average cost of testing per physician noting that Dr. Karev has the highest average cost.

Under physician, the user may sort by provider to see the breakdown of results classification per provider, the tests ordered by laboratory and a complete list of patients. The user chooses Alex Karev, and notes that only 3 tests were positive from this providers orders. If there are concerns he is ordering too many large panels and perhaps on inappropriate sets of patients, this value can be useful to track.

Under testing lab, the user wants to see which laboratory is providing results the fastest. The user scrolls down to see the average turn around time (TAT) by laboratory and note that GeneDx is returning results the fastest.

Advantages of the system may include: ease of navigation of a hierarchy of genetic conditions; a curated list of genes associated with the selected genetic conditions, a list of U.S.-based genetic panels matched based on the curated list of genes; a design that allows for ease of comparing several factors between laboratories and filter based on these factors; the ability to fill out order forms based on information stored in the software; the ability to send orders electronically directly to the laboratory: result being sent back electronically to the Navigator software; and analytics tools, including a list of genetic tests ordered by physician, by laboratory, by clinical guidance, which notes information about price and turnaround time (TAT).

### Example 8

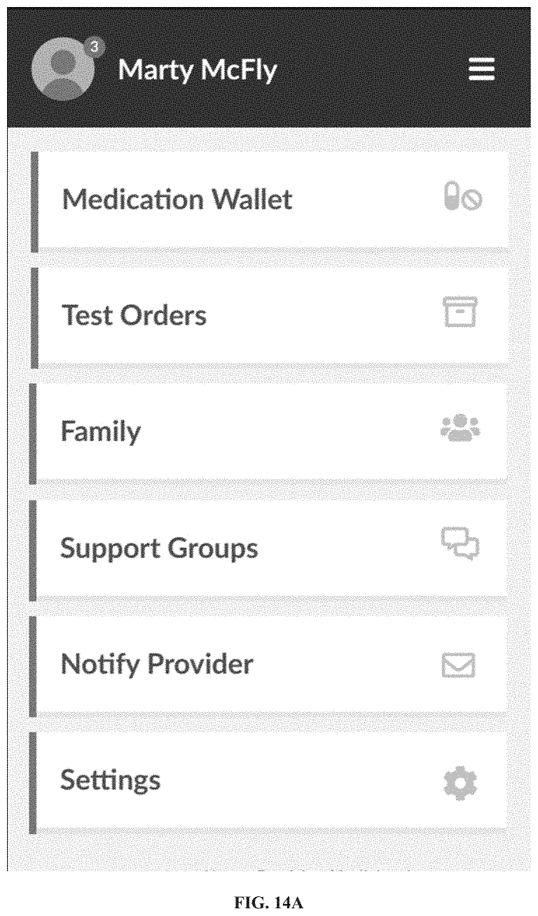

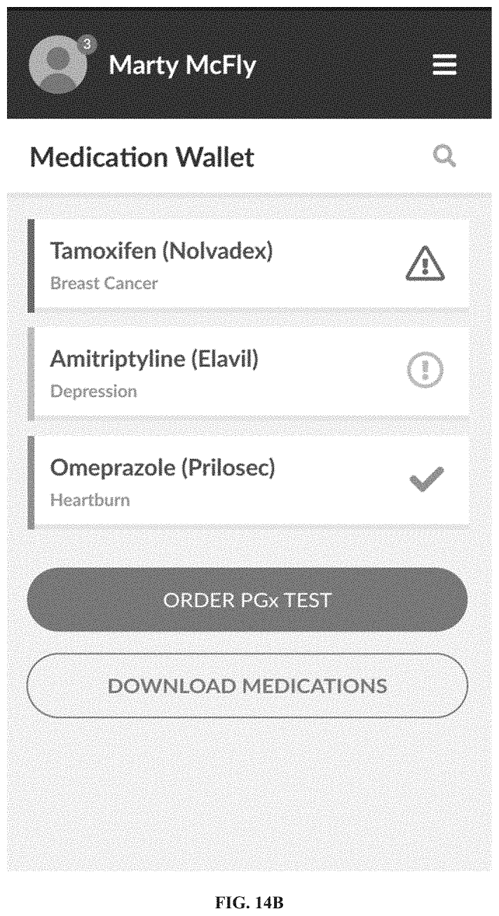

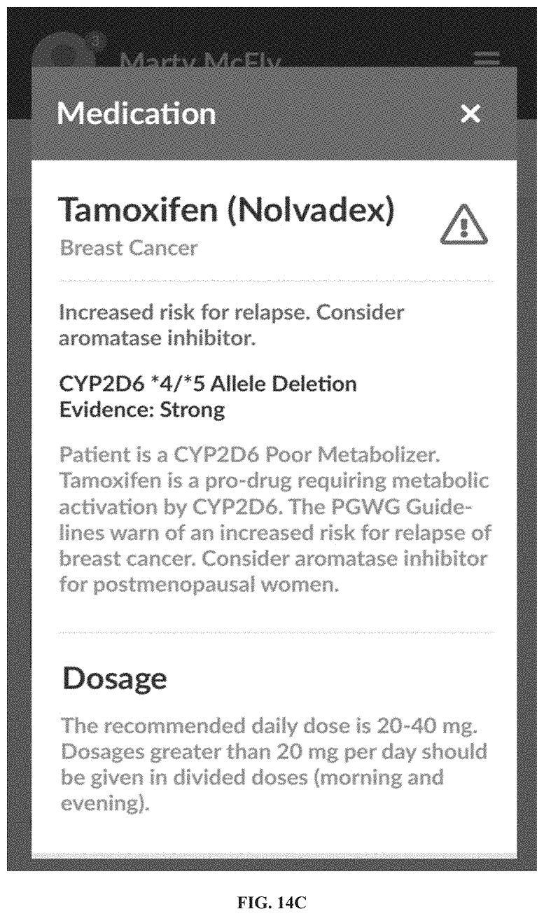

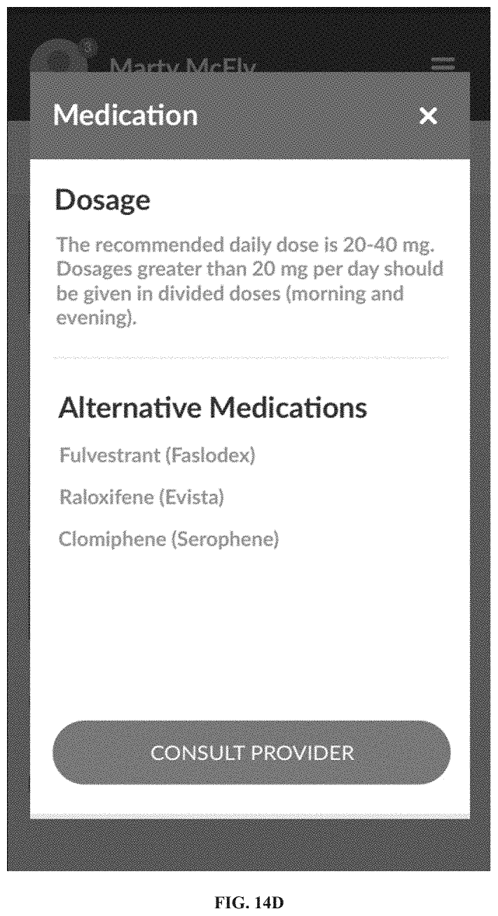

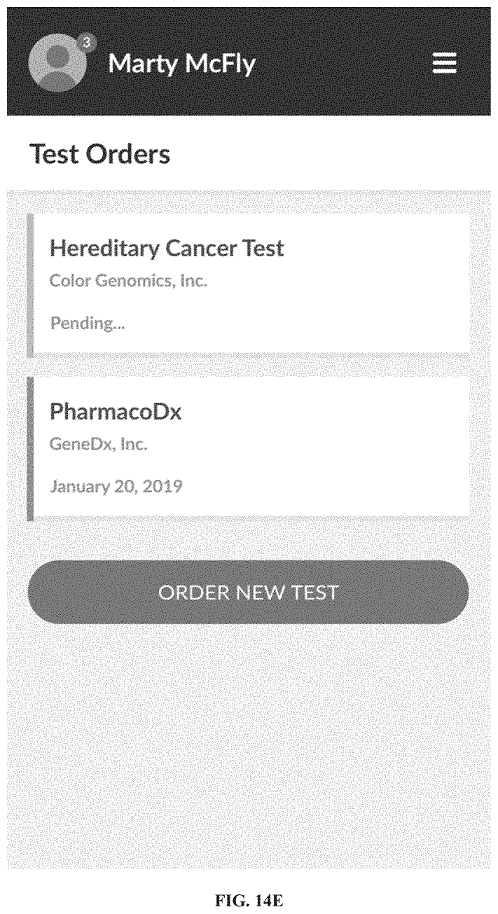

Using systems and methods of the present disclosure, the clinical guidance of genetic testing is provided to patients via a mobile application. FIGS. 14A-14E illustrate an example of screenshots of a mobile application to provide clinical guidance of genetic testing for patients, including a view of the main menu for the patient (FIG. 14A), a view of the medication wallet for the patient (FIG. 14B), a view of a specific medication for the patient (FIG. 14C), a view of a dosage of a medication for the patient (FIG. 14D), and a view of test orders for the patient (FIG. 14E), in accordance with disclosed embodiments.

The view of the main menu for the patient includes icons for selection of a medication wallet for the patient, test orders for the patient, family members of the patient, support group of the patient, notification of a provider of the patient, and settings (FIG. 14A).

The view of the medication wallet for the patient includes a list of one or more medications currently prescribed for and/or being taken by the patient for treatment of one or more conditions, diseases, or disorders (FIG. 14B). Examples include tamoxifen for breast cancer, amitriptyline for depression, and omeprazole for heartburn. The patient is able to click a link to order a pharmacogenomics (PGx) test or download a list of medications.

The view of a specific medication for the patient includes one or more warning symbols (such as a red exclamation mark in a triangle icon), one or more warning statements (e.g., increased risk for relapse), one or more gene associations with strength indication (e.g., strong evidence for an allele deletion), one or more medication suggestions (e.g., consider aromatase inhibitor for postmenopausal women), and a recommended dosage of the medication for the patient (FIG. 14C).

The view of a dosage of a medication for the patient includes a recommended dosage of the medication for the patient, and one or more alternative medications to treat a condition, disease, or disorder of the patient (FIG. 14D). The patient is able to click a link to consult his or her provider regarding any questions (e.g., questions about one or more medications).

The view of test orders for the patient includes a list of genetic tests that have been ordered for the patient, including the name of the genetic test (e.g., hereditary cancer test), the laboratory for the genetic test (e.g., Color Genomics, Inc.), and a status of the genetic test (e.g., pending or a date of completion) (FIG. 14E). The patient is able to click a link to order a new genetic test.

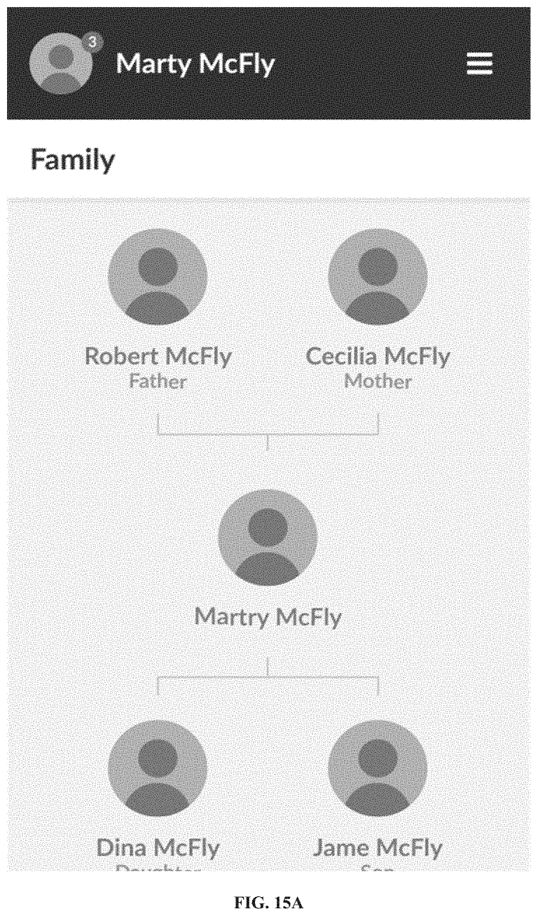

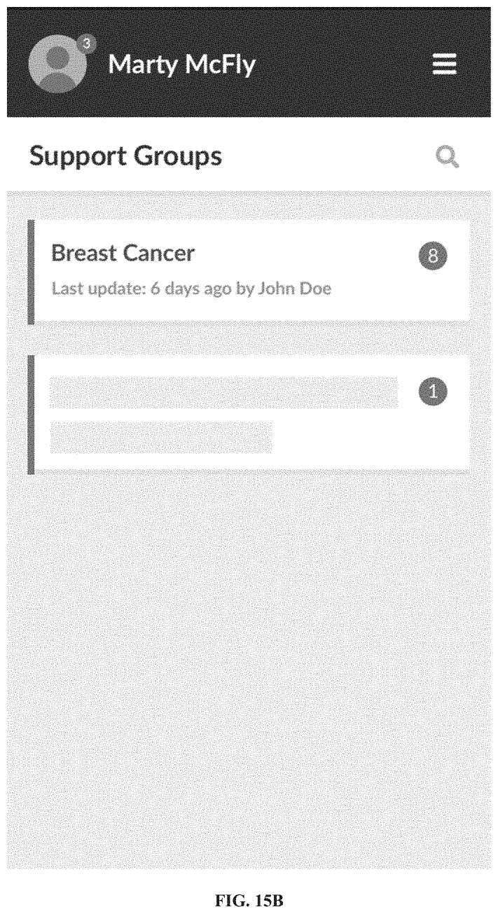

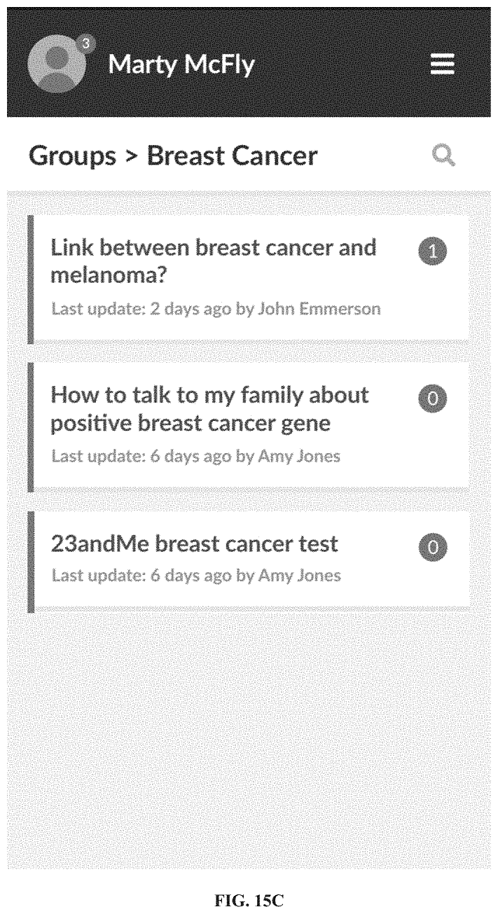

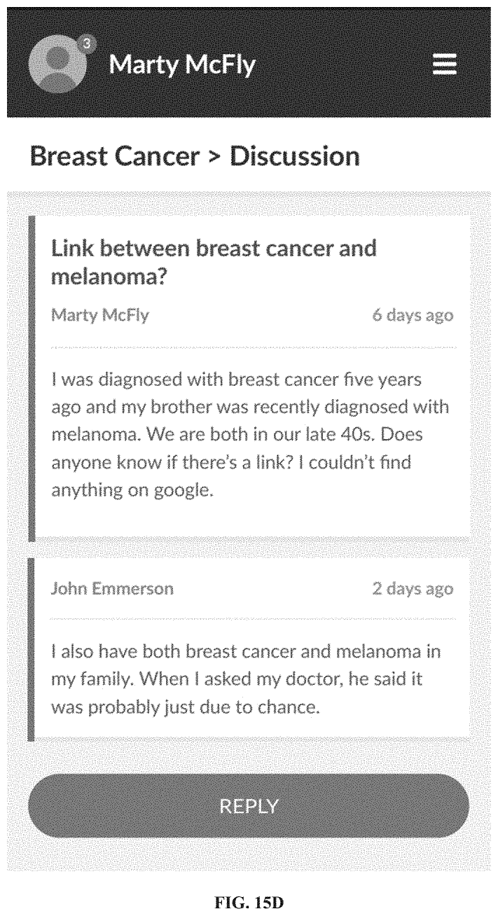

FIGS. 15A-15D illustrate an example of screenshots of a mobile application to provide clinical guidance of genetic testing for patients, including a view of the family members network for the patient (FIG. 15A), a view of the support groups for the patient (FIG. 15B), a view of a specific support group for the patient (FIG. 15C), and a view of a specific discussion in a support group for the patient (FIG. 15D), in accordance with disclosed embodiments.

The view of the family members network for the patient shows a list of family members of the patient, visualized as a family tree structure (FIG. 15A). For example, the family members network may include the parents of the patient and the children of the patient. The family members network may include other family members of the patient who are also users of the mobile application (e.g., who have been granted access by the patient to the patient's health information record). Alternatively, the family members network may include family members of the patient who are also patients obtaining clinical guidance.

The view of the support groups for the patient includes a list of support groups that the patient has joined (e.g., breast cancer), an indicator of when the last update to the support group was made, and an indicator of the number of new updates to the support group (FIG. 15B).

The view of a specific support group for the patient may include a name of the support group (e.g., corresponding to patients having a condition, disease, or disorder such as a breast cancer), a list of discussion thread topics, an indicator of when the last update to the support group was made, and an indicator of the number of new updates to the support group (FIG. 15C).

The view of a specific discussion in a support group for the patient may include a question posted by a user (e.g., the patient), the name of the user who posted the question or update, and a list of replies or updates posted by the patient or by other users or patients of the mobile application (FIG. 15D).

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A system for enabling an operator to generate a genomic health record of a subject, comprising:
a database comprising clinical indications;
a communications interface in communication with a computer of said operator; and
a mobile computing device comprising a computer processor operatively coupled to said database and said communications interface, wherein said computer processor is programmed to (i) receive a request from said mobile computing device of said operator to evaluate a clinical indication of said subject, (ii) upon receiving said request, determine genomic data relevant to said clinical indication, and (iii) generate an output, which output comprises a recommendation that said operator have said genomic data obtained for said subject.

2. The system of claim 1, wherein said operator is a physician.

3. The system of claim 1, wherein said computer processor is further programmed to navigate said operator to at least one of a clinical view, a disease category view, a disease view, and a genes view, subsequent to receiving said request from said computer of said operator to evaluate said clinical indication.

4. (canceled)

5. (canceled)

6. (canceled)

7. The system of claim 1, wherein said genomic data relevant to said clinical indication is determined based at least in part on information of said subject selected from the group consisting of clinical symptoms, patient history, and family history.

8. The system of claim 7, wherein said computer processor is further programmed to apply an artificial intelligence algorithm to said clinical indication and said information of said subject to determine said genomic data relevant to said clinical indication.

9. The system of claim 8, where said recommendation comprises one or more genetic tests to obtain said genomic data for said subject.

10. The system of claim 9, wherein said artificial intelligence algorithm comprises an optimization based on a constraint comprising one or more of: a number of said one or more genetic tests, a cost of said one or more genetic tests, an accuracy of said evaluation of said clinical indication, and a confidence of said evaluation of said clinical indication.

11. The system of claim 10, wherein said recommendation is customized to a hospital population.

12. The system of claim 11, wherein said hospital population is based on a geographical location.

13. The system of claim 1, wherein said mobile computing device is selected from the group consisting of a smart phone, a tablet computer, a smart watch, and a wearable computer.

14. (canceled)

15. (canceled)

16. (canceled)

17. (canceled)

18. A method for enabling an operator to generate a genomic health record of a subject, comprising:
(a) receiving a request from said operator via a mobile application to evaluate a clinical indication of said subject;
(b) upon receiving said request, determining genomic data relevant to said clinical indication; and
(c) generating an output, which output comprises a recommendation that said operator have said genomic data obtained for said subject.

19. The method of claim 18, wherein said operator is a physician.

20. The method of claim 18, further comprising navigating said operator to at least one of a clinical view, a disease category view, a disease view, and a genes view, subsequent to receiving said request from said computer of said operator to evaluate said clinical indication.

21. (canceled)

22. (canceled)

23. (canceled)

24. The method of claim 18, further comprising determining said genomic data relevant to said clinical indication based at least in part on information of said subject selected from the group consisting of clinical symptoms, patient history, and family history.

25. The method of claim 24, further comprising applying an artificial intelligence algorithm to said clinical indication and said information of said subject to determine said genomic data relevant to said clinical indication.

26. The method of claim 25, wherein said recommendation comprises one or more genetic tests to obtain said genomic data for said subject.

27. The method of claim 26, wherein said artificial intelligence algorithm comprises an optimization based on a constraint comprising one or more of: a number of said one or more genetic tests, a cost of said one or more genetic tests, an accuracy of said evaluation of said clinical indication, and a confidence of said evaluation of said clinical indication.

28. The method of claim 18, wherein said recommendation is customized to a hospital population.

29. The method of claim 28, wherein said hospital population is based on a geographical location.

30. (canceled)

31. The method of claim 18, wherein said mobile application is executed by a computer processor of a mobile computing device, wherein said mobile computing device is selected from the group consisting of a smart phone, a tablet computer, a smart watch, and a wearable computer.

32.-42. (canceled)
